(19) United States
(12) Patent Application Publication
(10) Pub. No.: US 2019/0358176 A1
(43) Pub. Date: Nov. 28, 2019

(54) METHODS OF SENSITIZING CANCER CELLS TO IMMUNE CELL KILLING

(71) Applicant: University of Pittsburgh - of the Commonwealth System of Higher Education, Pittsburgh, PA (US)

(72) Inventors: Andreas Vogt, Pittsburgh, PA (US); Christof Tobias Kaltenmeier, Pittsburgh, PA (US); Michael T. Lotze, Pittsburgh, PA (US); Waikok Michael Tsang, Pittsburgh, PA (US)

(73) Assignee: University of Pittsburgh - of the Commonwealth System of Higher Education, Pittsburgh, PA (US)

(21) Appl. No.: 16/513,194

(22) Filed: Jul. 16, 2019

Related U.S. Application Data
(63) Continuation of application No. PCT/US2018/016230, filed on Jan. 31, 2018.
(60) Provisional application No. 62/452,856, filed on Jan. 31, 2017.

(51) Int. Cl. A61K 31/135; A61K 39/395; A61K 35/17; A61K 38/20; A61K 38/21; A61P 35/00

(52) U.S. Cl. CPC A61K 31/135; A61K 39/3955; A61P 35/00; A61K 38/2013; A61K 38/212; A61K 35/17

(57) ABSTRACT

The presently disclosed subject matter is directed to dual specificity mitogen-activated protein kinase phosphatase (DUSP-MKP) inhibitors that sensitize cancer cells immune cell killing and methods of using the disclosed DUSP-MKP inhibitors for the treatment of cancer.

### PRIORITY

This application claims priority to U.S. Provisional Application Ser. 62/452,856, filed Jan. 31, 2017, which is incorporated by reference herein in its entirety.

### GRANT INFORMATION

This invention was made with government support under grant numbers CA147985, HD053287, CA181450, and CA047904 awarded by the National Institutes of Health and grant number W911NF-14-1-0422 awarded by the Army/ARL. The government has certain rights in the invention.

### 1. INTRODUCTION

The presently disclosed subject matter relates to the administration of a DUSP-MKP inhibitor for the treatment of a cancer, and also to the administration of a DUSP-MKP inhibitor in combination with a cancer therapy/immunotherapy agent for the treatment of a cancer.

### 2. BACKGROUND OF THE INVENTION

Mitogen-activated protein kinase phosphatases (MKPs) are a subgroup of the dual specificity phosphatase (DUSP) family that has recently been termed DUSP-MKPs to reconcile both current gene nomenclature and historical denominations (1). DUSP-MKPs dephosphorylate and inactivate the mitogen-activated protein kinases ERK, JNK/SAPK, and p38 on tyrosine and threonine residues, thereby regulating duration and amplitude of mitogenic and survival signaling (2). A large body of literature that has been subject to multiple comprehensive reviews supports a role of DUSP-MKPs in cancer (1, 3, 4). The prototypic DUSP-MKP, DUSP1/MKP-1 is overexpressed in prostate, gastric, breast, pancreatic, ovarian, non-small cell lung (NSCLC), and metastatic colorectal cancer, and has been associated with decreased progression-free survival (5, 6). Genetic depletion of MKP-1 by siRNA enhances sensitivity of cancer cells to clinically used antineoplastic agents (7, 8) whereas its overexpression promotes chemoresistance (9). In mice, genetic ablation of DUSP1/MKP-1 limits the tumorigenicity of pancreatic cancer cells (8) and inhibits non-small cell lung cancer tumorigenesis and metastasis (10). Small molecule inhibitors of DUSP-MKPs could therefore provide novel approaches to treat cancer.

The discovery of potent and selective inhibitors of DUSPs, however, has been hindered by a high degree of conservation between their active sites, a shallow and feature-poor topology (2), and the presence of a reactive, active site cysteine, which is critical for enzymatic activity but sensitive to oxidation. Perhaps not too surprisingly, in vitro screens for DUSP inhibitors have yielded agents that were reactive chemicals or lacked biological activity. The utility of DUSP-MKP inhibitors as therapeutics is also disputed because of the varied roles that DUSP-MKPs play in physiology and pathophysiology, and their overlapping substrate specificities (2). Consequently, this class of enzymes is often thought of as “undruggable”.

Using a zebrafish live reporter for fibroblast growth factor (FGF) activity, a biologically active inhibitor of zebrafish Dusp6/Mkp3, (E)-2-benzylidene-3-(cyclohexylamino)-2,3-dihydro-1H-inden-1-one (BCI), was identified (11). Subsequent in vivo structure activity relationship (SAR) studies in zebrafish embryos coupled with mammalian cell-based assays for inhibition of DUSP1/MKP-1 and DUSP6/MKP-3 using 33 structural congeners identified an analog (BCI-215) that retained FGF hyperactivating and cellular DUSP6/MKP-3 and DUSP1/MKP-1 inhibitory activity, but was non-toxic to zebrafish embryos and an endothelial cell line (12).

### 3. SUMMARY OF THE INVENTION

The presently disclosed subject matter relates to methods of treating a cancer in which a dual specificity mitogen-activated protein kinase phosphatase (DUSP-MKP) inhibitor is used to sensitize cancer cells to immune cell killing. In certain embodiments, the presently disclosed subject matter can be used to improve, increase, or enhance the anti-cancer response of a subject by administering, to the subject, a DUSP-MKP inhibitor that sensitizes cancer cells to immune cell killing, together with an agent that promotes immune cell killing of cancer cells, for example, but not by limitation, by sensitizing cancer cells to lymphokine-activated killer (“LAK”) cell activity.

In certain embodiments, the subject has been determined to exhibit an inadequate anti-cancer response to checkpoint inhibitor therapy (“inhibitor monotherapy,” administered without a DUSP-MKP inhibitor), either by, for example, clinical history of the individual subject, by correlation to one or more biomarker, or by the cancer type involved. In certain non-limiting embodiments, treatment with the DUSP-MKP inhibitor can be instituted prior to treatment with the agent that promotes cell killing and the two types of therapy can or cannot overlap in time. In certain embodiments, treatment with the DUSP-MKP inhibitor can be administered concurrently with the agent that promotes cell killing.

In certain embodiments, the presently disclosed subject matter provides for a method of treating cancer in a subject comprising administering, to the subject, (i) an amount of a dual specificity mitogen-activated protein kinase phosphatase (DUSP-MKP) inhibitor that sensitizes cancer cells to immune cell killing and (ii) an agent that promotes immune cell killing, for example, a cell-mediated anti-cancer immune response in the subject.

In certain embodiments, the immune cell killing is immunogenic cell death (“ICD”). In certain embodiments, treatment with DUSP-MKP inhibitor renders them more sensitive to lymphokine-activated killer cell activity.

In certain embodiments, the agent that promotes immune cell killing can be a checkpoint inhibitor, for example, but not limited to, an antibody selected from the group consisting of an antibody for CTLA-4 (for example, ipilimumab), an antibody for PD-1 (for example, pembrolizumab, nivolumab, or BGB-A137), and an antibody for PD-L1 (for example, atezolizumab, avelumab, ordurvalumab). In certain non-limiting embodiments, the agent is an antibody for CD52 (for example, alemtuzumab), and an antibody for CD20 (for example, ofatumumab or rituximab).

In certain embodiments, the agent that promotes immune cell killing can comprise immune cells selected from the group consisting of natural killer cells and dendritic cells, wherein the immune cells are activated in vitro and introduced to the subject. Alternatively or additionally the immune cells can comprise T cells or interleukin-2 (IL-2)-activated peripheral blood mononuclear cells (PBMCs). Said cells can be autologous or heterologous.

In certain embodiments, the agent that promotes immune cell killing can be a lymphokine, for example but not limited to interleukin-2 or interferon alpha.

In certain embodiments, the presently disclosed subject matter provides for a method of treating cancer in a subject comprising:

(i) determining whether the subject expresses cancer cells that are resistant to treatment with an inhibitor monotherapy, wherein the resistant cells treated with the inhibitor monotherapy exhibit DUSP-MKP activity; and

(ii) where the subject expresses cancer cells that are resistant to treatment with the inhibitor monotherapy, treating the subject with a first agent comprising a DUSP-MKP inhibitor that sensitizes cancer cells to immune cell killing or a combination of the first agent comprising a DUSP-MKP inhibitor that sensitizes cancer cells to immune cell killing with a second agent that that promotes a cell-mediated anti-cancer immune response.

In certain embodiments, the presently disclosed subject matter provides for a method of treating cancer in a subject comprising administering to the subject in need thereof an effective amount of (i) a first agent that inhibits DUSP6-induced dephosphorylation of extracellular signal-related kinase (ERK) and sensitizes cancer cells to immune cell killing and (ii) a second agent that promotes immune cell killing.

In certain embodiments, the presently disclosed subject matter provides for a method for reducing cancer cell proliferation or promoting cancer cell death in a subject in need thereof comprising administering to the subject an effective amount of (i) a first agent comprising a DUSP-MKP inhibitor that sensitizes cancer cells to immune cell killing and (ii) a second agent that promotes immune cell killing.

In certain embodiments, the presently disclosed subject matter provides for a method for reducing cancer cell proliferation or promoting cancer cell death in a subject in need thereof comprising contacting a cancer cell of the subject with an effective amount of (i) a first agent comprising a DUSP-MKP inhibitor that sensitizes cancer cells to immune cell killing and (ii) a second agent that promotes immune cell killing.

In certain embodiments, the presently disclosed subject matter provides for a method for reducing cancer cell proliferation or promoting cancer cell death in a subject in need thereof comprising contacting a cancer cell of the subject with (i) a first agent comprising a compound having the formula:

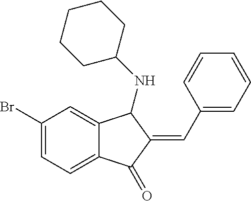 [SMILES: O=C1/C(=C/c2ccccc2)C(NC2CCCCC2)c2cc(Br)ccc21]

or an analog thereof, in an amount effective to increase levels of phosphorylated ERK or to decrease levels of de-phosphorylated ERK in the cancer cell and sensitize the cancer cell to immune cell killing and (ii) a second agent that promotes immune cell killing.

In certain embodiments, the presently disclosed subject matter provides for a method of inhibiting cancer cell metastasis in a subject in need thereof comprising administering to a subject in need thereof an effective amount of (i) a first agent comprising a dual specificity mitogen-activated protein kinase phosphatase (DUSP-MKP) inhibitor that sensitizes cancer cells to immune cell killing and (ii) a second agent that promotes immune cell killing.

In certain embodiments, the presently disclosed subject matter provides for a kit comprising: (i) one or more agent that can (a) decrease/inhibit the activity of DUSP6; (b) decrease the activity DUSP6 and DUSP1; (c) sensitize cancer cells to immune cell killing; and (d) reduce or inhibit cancer cell and/or tumor cell growth and (ii) one or more agent that can promote immune cell killing.

In certain embodiments, the presently disclosed subject matter provides for a kit comprising a container comprising: (i) an effective amount of a first agent comprising a DUSP-MKP inhibitor comprising BCI-215 or an analog thereof that sensitizes cancer cells to immune cell killing; (ii) an effective amount of a second agent that promotes immune cell killing; and (iii) a pharmaceutically acceptable buffer.

### 4. BRIEF DESCRIPTION OF FIGURES

FIG. 1. Structures of compounds used in this study. The study comprises comparative evaluations of three previously described DUSP inhibitors (NSC95397; sanguinarine, (E)-2-benzylidene-3-(cyclohexylamino)-2,3-dihydro-1H-inden-1-one (BCI), its non-toxic analog (BCI-215), and menadione (vitamin K3) as a positive control for hepatotoxicity). MAPK inhibitors used for pathway evaluation SCH772984, SB203580, SP600125 and JNK-IN-08 were from commercial sources.

FIG. 2A-2E. BCI-215 is non-toxic to rat hepatocytes and developing zebrafish embryos. (A-C) Rat hepatocytes were treated in 96 well plates with ten point concentration gradients of DUSP inhibitors and menadione as a positive control for hepatotoxicity. Sanguinarine, NSC95397, BCI, and menadione, but not BCI-215, produced dose-dependent cell death in rat hepatocytes as measured by (A) propidium iodide (PI) uptake and (B) loss of mitochondrial membrane integrity. (C) Hepatocyte toxicity correlated with production of reactive oxygen species (ROS). (D) and (E) In contrast to other DUSP inhibitors, BCI-215 did not generate ROS in developing zebrafish embryos. Data and images are from a single experiment that has been repeated once.

FIG. 3A-3E. BCI-215 inhibits motility, survival, and metastatic outgrowth of human breast cancer cells. (A-C) MDA-MB-231 cells were plated in the wells of an Oris™ Pro 384 cell migration plate, stained with PI and Hoechst 33342 48 h thereafter, and analyzed by high-content analysis for cells that had migrated into the exclusion zone (cell migration), cell loss (cell density), necrosis (% PI positive cells), and nuclear shrinkage (nucleus area). Each data point is the mean of four technical replicates ±SEM from a single experiment that has been repeated four times. All agents inhibited cancer cell migration and caused cell loss with IC50s between 7-15 μM. BCI-215 showed no signs of necrosis at antimigratory and cytotoxic concentrations. (D) MDA-MB-231 cells carrying a mitochondrial-targeted, GFP-labeled cytochrome C biosensor were seeded on a layer of matrigel and treated with BCI-215 the next day (Tx). After two days of exposure, drug was washed out and cells allowed to grow for an additional three to five days. Z-stacks were acquired at the indicated time points and cell numbers calculated from maximum projection images. At the end of the study (day 6-8), cells were incubated with PI and the percentage of PI positive cells determined. (E) BCI-215 inhibits colony formation and causes pronounced secondary cell lysis in the six-day colony formation assay. Data are the averages ±SEM of three independent experiments, each performed in triplicate.

FIG. 4A-4B. (A) Short-term toxicity and motility inhibition on collagen-coated plastic. MDA-MB-231 cells (15,000/well) were plated in the wells of an Oris™ Pro 384 cell migration plate and stained with PI and Hoechst 33342 48 h thereafter. Images show the bottom left quarter of an entire microwell, acquired on the ArrayScan II at 5×, and demonstrate closure of the cell exclusion zone (bare area in the upper right hand corner), cell density (Hoechst stained nuclei in blue), and PI positive cells (red). Scale bar, 300 μm. (B) Toxicity in matrigel six days after treatment with BCI-215. MDA-MB-231 cells (2000/well) transduced with a biosensor consisting of EGFP with a mitochondrial targeting sequence derived from cytochrome-C oxidase subunit VIII were plated on a cushion of matrigel and treated with vehicle or BCI-215. After two days, the medium was replaced and cells were allowed to recover for 4 days. Images show GFP/PI overlays of collapsed Z-stacks (20 planes, 5 μm) acquired at 20× magnification on the ImageXpress Ultra. Scale bar, 200 μm.

FIG. 5A-5D. BCI and BCI-215 cause apoptotic cell death at concentrations that induce ERK phosphorylation. MDA-MB-231 cells were treated with vehicle (DMSO), BCI, or BCI-215 and stained with Hoechst 33342 and anti-phospho-ERK and anti-cleaved caspase-3 antibodies, respectively. (A) Fluorescence micrographs show pyknotic nuclei indicative of early apoptosis. Images are maximum projections of a ten plane, 0.25 μm each z-series acquired using a 60× objective on a Molecular Devices ImageXpress Ultra high content reader. BCI and BCI-215 were at 22 μM. (B) Multiparametric analysis of chromatin condensation, caspase-3 cleavage, and ERK phosphorylation by high-content analysis. Each box plot is the aggregate of four (caspase) or five (nuclear condensation and ERK phosphorylation) independent experiments. Boxes show upper and lower quartiles; whiskers, range; dot, mean. *, p<0.05; **, p<0.01; ****, p<0.001 vs. DMSO by one-way ANOVA with Dunnett's multiple comparison test. The last data point for cleaved caspase is an n=3 for 50 μM BCI-215 with two of the three values being identical. (C and D) Confirmation of apoptosis with secondary cell lysis by flow cytometry. Data in (D) are the averages ±SEM of three independent experiments. Early apoptosis, Q3, Annexin V positive and PI negative; late apoptosis, Q2, Annexin V and PI positive; necrosis (Q1, PI positive, Annexin V negative.

FIG. 6A-6B. (A) BCI-215 sensitizes breast cancer cells to immune cell kill. MDA-MB-231 cells were treated overnight in 384 well plates with vehicle or 3 μM BCI-215 followed by washout. Cells were subsequently exposed to various ratios of PBMC-derived LAK. After 24 hours, cells were fixed and stained with Hoechst 33342. Cells were imaged on the ArrayScan II, cancer cell nuclei identified and gated by their larger size compared with PBMC, and enumerated. Cell densities were normalized to vehicle or BCI-215 in the absence of activated immune cells, respectively. Data are the averages ±SEM from four independent experiments, each performed in triplicate. (B) Comparison of BCI-215 vs. clinically used antineoplastic agents doxorubicin (DOX) and cisplatin (CDDP). MDA-MB_231 cells were either stained with CellTracker green or transduced with a mitochondrial-targeted, GFP-labeled cytochrome C biosensor and processed and analyzed as in (A) except that cancer cells were specifically identified by green fluorescence instead of nucleus size gating. Each data point represents the mean±SEM of three independent experiments.

FIG. 7A-7D. BCI-215 activates mitogen- and stress-activated protein kinase cascades in the absence of oxidative stress. (A) Activation kinetics. MDA-MB-231 human breast cancer cells were treated with BCI or BCI-215 (20 μM) for the indicated time points and analyzed for phosphorylation of the DUSP1/MKP-1 and DUSP6/MKP-3 substrates, ERK, JNK/SAPK, and p38, as well as their upstream activators MEK1 and MKK4/SEK1 by Western blot. (B) Activation of kinase cascades in three different cell lines. Cells were treated for 1 hour with vehicle (DMSO) 20 μM BCI-215 (215), or 5 doxorubicin (DOX). Data are from a single experiment that has been repeated once. (C and D) ROS generation. MDA-MB-231 cells were pre-labeled with Hoechst 33342 and chloromethyl-fluorescein diacetate, acetyl ester (CM-H2-DCFDA) for 30 min followed by treatment with test agents for up to 5 hours. (C) At the indicated time points, cells were imaged and the percentage of ROS positive enumerated. (D) Concentration response at the 2 hour time point. Data in both panels are from single experiments that have been repeated twice. Each data point is the mean of four wells ±SEM from a single experiment that has been repeated twice.

FIG. 8A-8E. Effect of MAPK inhibition of BCI-215 toxicity. MDA-MB-231 cells were pre-treated with concentration gradients of MAPK inhibitors followed by vehicle or a toxic concentration of BCI-215 (25 After 24 hours, cells were stained with Hoechst 33342 and an antibody against cleaved caspase-3, and analyzed for (A) cell density (B and C) nuclear morphology, and (D) caspase cleavage. Data on graphs depict % rescue from BCI, calculated as 1−((data point−DMSO)/(DMSO-BCI-215))*100. Images in (E) illustrate cell loss and nuclear morphology with vehicle (DMSO) and BCI-215 alone, or of BCI-215 in the presence of SCH771984 (375 nM), SB203580 (18 SP600125 (18 or JNK-IN-8 (1.8 Data are the averages of 4-7 independent experiments ±SEM, each performed in quadruplicate. Images are from an ArrayScan VTI using a 20× objective.

FIG. 9. Reduced toxicity of BCI-215 in the presence of doxorubicin. MDA-MB-231 carrying the mitochondrial-targeted, GFP-labeled cytochrome C biosensor were treated for 2-3 hours with vehicle (DMSO) or doxorubicin (5 μM) before being exposed to concentration gradients of BCI-215 for 20 h. Plates were scanned live on an ImageXpress high content reader at 20× magnification and GFP positive cells enumerated. Cell densities were normalized to vehicle or doxorubicin alone, respectively. Data are the averages ±SD from three independent experiments, each performed in quadruplicate.

### 5. DETAILED DESCRIPTION OF THE INVENTION

For clarity of description, and not by way of limitation, the detailed description of the presently disclosed subject matter is divided into the following subsections:

(i) Definitions;

(ii) DUSP-MKP inhibitors and Pharmaceutical Compositions;

(iii) Methods of treatment; and

(iv) Kits.

### 5.1 Definitions

The terms used in this specification generally have their ordinary meanings in the art, within the context of the presently disclosed subject matter and in the specific context where each term is used. Certain terms are discussed below, or elsewhere in the specification, to provide additional guidance to the practitioner in describing the formulations and methods of the presently disclosed subject matter and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, a “protein” or “polypeptide” refers to a molecule comprising at least one amino acid residue.

As used herein, the term “analog” refers to a structurally related polypeptide or nucleic acid molecule having the function of a reference polypeptide or nucleic acid molecule.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, prevents, decreases, suppresses, eliminates or blocks) the signaling function of a protein or pathway. An inhibitor can be any compound or molecule that changes any activity of a named protein (signaling molecule, any molecule involved with the named signaling molecule or a named associated molecule), such as DUSP, or interferes with the interaction of a named protein, e.g., DUSP, with signaling partners. Inhibitors also include molecules that indirectly regulate the biological activity of a named protein, e.g., DUSP, by intercepting upstream signaling molecules.

The terms “inhibiting,” “eliminating,” “decreasing,” “reducing” or “preventing,” or any variation of these terms, referred to herein, includes any measurable decrease or complete inhibition to achieve a desired result.

As used herein, the term “contacting” cancer cells (or a tumor) with a compound or molecule (e.g., one or more inhibitors, activators and/or inducers) refers to placing the compound in a location that will allow it to touch the cell (or the tumor). The contacting may be accomplished using any suitable methods. For example, contacting can be accomplished by adding the compound to a collection of cells, e.g., contained with a tube or dish. Contacting may also be accomplished by adding the compound to a culture medium comprising the cells. Contacting may also be accomplished by administering a compound to a subject that has one or more cancer cells, even where the site of administration is distant from the location of the cancer cell(s), provided that the compound would reasonably be expected access to the cancer cell(s), for example, by circulation through blood, lymph or extracellular fluid.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “treating” or “treatment” (and grammatical variations thereof such as “treat”) refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing and/or inhibiting metastases, reducing cancer cell proliferation, promoting cancer cell death, decreasing the rate of disease progression, amelioration or palliation of the disease state and remission or improved prognosis. By preventing progression of a disease or disorder, a treatment can prevent deterioration due to a disorder in an affected or diagnosed subject or a subject suspected of having the disorder, but also a treatment can prevent the onset of the disorder or a symptom of the disorder in a subject at risk for the disorder or suspected of having the disorder. In certain embodiments, “treatment” can refer to a decrease in the severity of complications, symptoms and/or cancer or tumor growth. For example, and not by way of limitation, the decrease can be a decrease of about 10%, about 20%, about 30%, about 40%, about 50%, about 60%, about 70%, about 80%, about 90%, about 95%, about 98% or about 99% in severity of complications, symptoms and/or cancer or tumor growth, for example relative to a comparable control subject not receiving the treatment. In certain embodiments, “treatment” can also mean prolonging survival as compared to expected survival if treatment is not received.

An “effective amount” (or “therapeutically effective amount”) is an amount sufficient to affect a beneficial or desired clinical result upon treatment. In certain embodiments, a therapeutically effective amount refers to an amount that is able to achieve one or more of an anti-cancer effect, prolongation of survival and/or prolongation of period until relapse. For example, and not by way of limitation, a therapeutically effective amount can be an amount of a compound (e.g., inhibitor) that produces an “anti-cancer effect.” A therapeutically effective amount can be administered to a subject in one or more doses. The therapeutically effective amount is generally determined by the physician on a case-by-case basis and is within the skill of one in the art. Several factors are typically taken into account when determining an appropriate dosage to achieve a therapeutically effective amount. These factors include age, sex and weight of the subject, the condition being treated, the severity of the condition and the form and effective concentration of the cells administered.

An “anti-cancer effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, a reduction in tumor growth rate and/or a reduction in tumor metastasis, and/or an increase in cancer cell death and/or an increase in cancer cell apoptosis. In certain embodiments, an anti-cancer effect can refer to a complete response, a partial response, a stable disease (without progression or relapse), a response with a later relapse or progression-free survival in a patient diagnosed with cancer.

An “anti-cancer agent” or “agent”, as used herein, can be any agent, molecule, compound, chemical or composition that has an anti-cancer effect. Anti-cancer agents include, but are not limited to, chemotherapeutic agents, radiotherapeutic agents, cytokines, anti-angiogenic agents, apoptosis-inducing agents, and anti-cancer antibodies.

### 5.2 DUSP-MKP Inhibitors and Pharmaceutical Compositions

The presently disclosed subject matter relates to the administration of one or more dual specificity mitogen-activated protein kinase phosphatase (DUSP-MKP) inhibitors for the treatment of cancer. In certain non-limiting embodiments, the presently disclosed subject matter discloses a DUSP-MKP inhibitor is (E)-2-benzylidene-3-(cyclohexylamino)-2,3-dihydro-1H-inden-1-one (BCI) or an analog thereof, for example, BCI-215. Non-limiting examples of such DUSP-MKP inhibitors are set forth in references (11), (12) and (13) below and, for example, in U.S. Pat. Nos. 9,127,016 and 9,439,877, as well as U.S. patent application Ser. No. 15/243,089 (Publication No. US2016/0355459), each and all of which are incorporated by reference in their entireties herein.

In certain embodiments, the DUSP-MKP inhibitor comprises a compound having the formula or an analog or prodrug thereof:

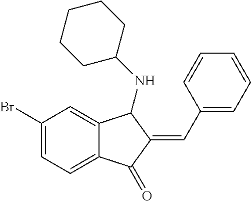 [SMILES: O=C1/C(=C/c2ccccc2)C(NC2CCCCC2)c2cc(Br)ccc21]

In certain embodiments, BCI-215 has the general formula:

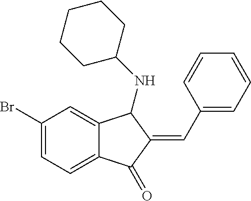

In certain non-limiting embodiments, the presently disclosed subject matter provides for pharmaceutical formulations comprising one or more DUSP-MKP inhibitors disclosed herein for therapeutic use. In certain embodiments, the pharmaceutical formulation comprises one or more DUSP-MKP inhibitor and a pharmaceutically acceptable carrier.

“Pharmaceutically acceptable carrier,” as used herein, includes any carrier which does not interfere with the effectiveness of the biological activity of the active ingredients, e.g., inhibitors, and that is not toxic to the patient to whom it is administered. Non-limiting examples of suitable pharmaceutical carriers include phosphate-buffered saline solutions, water, emulsions, such as oil/water emulsions, various types of wetting agents and sterile solutions. Additional non-limiting examples of pharmaceutically acceptable carriers can include gels, bioadsorbable matrix materials, implantation elements containing the inhibitor and/or any other suitable vehicle, delivery or dispensing means or material. Such carriers can be formulated by conventional methods and can be administered to the subject. In certain embodiments, the pharmaceutical acceptable carrier can include buffers such as phosphate, citrate, and other organic acids; antioxidants including ascorbic acid and methionine; preservatives (such as, but not limited to, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, a suitable pharmaceutically acceptable carrier can include one or more of water, saline, phosphate buffered saline, dextrose, glycerol, ethanol or combinations thereof.

In certain embodiments, the pharmaceutical formulations of the presently disclosed subject matter can be formulated using pharmaceutically acceptable carriers well known in the art that are suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. For example, and not by way of limitation, formulations of the presently disclosed subject matter can be administered to the patient intravenously in a pharmaceutically acceptable carrier such as physiological saline.

In certain embodiments, the methods and formulations of the present invention can be used for reducing, inhibiting, preventing or reversing cancer and/or tumor growth. Standard methods for intracellular delivery can be used (e.g., delivery via liposome).

In certain non-limiting embodiments, the pharmaceutical compositions of the presently disclosed subject matter can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral or nasal ingestion by a patient to be treated. In certain embodiments, the pharmaceutical formulation can be a solid dosage form. In certain embodiments, the tablet can be an immediate release tablet. In certain embodiments, the tablet can be an extended or controlled release tablet. In certain embodiments, the solid dosage can include both an immediate release portion and an extended or controlled release portion.

In certain embodiments, the pharmaceutical compositions comprise a DUSP-MKP inhibitor that sensitizes cancer cells to immune cell killing, together with an agent that promotes immune cell killing of cancer cells. In certain embodiments, the agent that promotes immune cell killing of cancer cells, promotes a cell-mediated anti-cancer immune response in the subject. In certain embodiments, the immune cell killing is immunogenic cell death (“ICD”). In certain embodiments, treatment with DUSP-inhibitor renders them more sensitive to lymphokine-activated killer cell activity.

In certain embodiments, the agent that promotes immune cell killing of cancer cells is an agent that sensitizes cancer cells to lymphokine-activated killer (“LAK”) cell activity. In certain embodiments, the agent that promotes immune cell killing of cancer cells is a checkpoint inhibitor. In certain embodiments, a checkpoint inhibitor is an antibody selected from the group consisting of an antibody for CTLA-4 (for example, ipilimumab), an antibody for PD-1 (for example, pembrolizumab, nivolumab, or BGB-A137), and an antibody for PD-L1 (for example, atezolizumab, avelumab, ordurvalumab). In certain embodiments, the agent that promotes immune cell activity is an antibody for CD52 (for example, alemtuzumab), and an antibody for CD20 (for example, ofatumumab or rituximab).

Examples of inhibitors include, but are not limited to, compounds, molecules, chemicals, polypeptides and proteins that inhibit and/or reduce the expression and/or activity of the protein encoded by a DUSP gene. Alternatively or additionally, the inhibitor can include compounds, molecules, chemicals, polypeptides and proteins that inhibit and/or reduce the expression and/or activity of one or more downstream targets of the DUSP gene.

Additional non-limiting examples of inhibitors include ribozymes, antisense oligonucleotides, shRNA molecules and siRNA molecules that specifically inhibit or reduce the expression and/or activity of a DUSP gene and/or inhibit or reduce the expression and/or activity of one or more downstream targets of a DUSP gene. One non-limiting example of an inhibitor comprises an antisense, shRNA or siRNA nucleic acid sequence homologous to at least a portion of a DUSP gene sequence, wherein the homology of the portion relative to the DUSP gene sequence is at least about 75 or at least about 80 or at least about 85 or at least about 90 or at least about 95 or at least about 98 percent, where percent homology can be determined by, for example, BLAST or FASTA software.

In certain non-limiting embodiments, the complementary portion may constitute at least 10 nucleotides or at least 15 nucleotides or at least 20 nucleotides or at least 25 nucleotides or at least 30 nucleotides and the antisense nucleic acid, shRNA or siRNA molecules may be up to 15 or up to 20 or up to 25 or up to 30 or up to 35 or up to 40 or up to 45 or up to 50 or up to 75 or up to 100 nucleotides in length. Antisense, shRNA or siRNA molecules can comprise DNA or atypical or non-naturally occurring residues, for example, but not limited to, phosphorothioate residues and locked nucleic acids.

In certain embodiments, an inhibitor can include an antibody, or a derivative thereof, that specifically binds to and inhibits and/or reduces the expression and/or activity of the protein that is encoded by the DUSP gene, e.g., an antagonistic antibody. Alternatively or additionally, an inhibitor can include an antibody, or derivative thereof, that specifically binds to and inhibits and/or reduces the expression and/or activity of one or more downstream targets of the DUSP gene.

The phrase “specifically binds” refers to binding of, for example, an antibody to an epitope or antigen or antigenic determinant in such a manner that binding can be displaced or competed with a second preparation of identical or similar epitope, antigen or antigenic determinant. Non-limiting examples of antibodies, and derivatives thereof, that can be used in the disclosed methods include polyclonal or monoclonal antibodies, chimeric, human, humanized, primatized (CDR-grafted), veneered or single-chain antibodies, phase produced antibodies (e.g., from phage display libraries), as well as functional binding fragments of antibodies. Antibody binding fragments, or portions thereof, include, but are not limited to, Fv, Fab, Fab′ and F(ab′)₂. Such fragments can be produced by enzymatic cleavage or by recombinant techniques.

In certain embodiments, the agent that promotes immune cell killing can comprise immune cells selected from the group consisting of natural killer cells and dendritic cells, wherein the immune cells are activated in vitro and introduced to the subject. In certain embodiments, the immune cells can comprise T cells or interleukin-2 (IL-2)-activated peripheral blood mononuclear cells (PBMCs). In certain embodiments, the immune cells can be autologous. In certain embodiments, the cells can be heterologous. In certain embodiments, the agent that promotes immune cell killing can be a lymphokine, for example but not limited to interleukin-2 or interferon alpha.

In certain non-limiting embodiments, treatment with the DUSP-MKP inhibitor can be instituted prior to treatment with the agent that promotes cell killing and the two types of therapy can or may not overlap in time. In certain embodiments, treatment with the DUSP-MKP inhibitor can be administered concurrently with the agent that promotes cell killing.

In certain embodiments, the presently disclosed subject matter provides a pharmaceutical composition comprising a DUSP-MKP inhibitor and/or a PI3Kα inhibitor. In certain embodiments, the presently disclosed subject matter provides a parenteral composition comprising a DUSP-MKP inhibitor and/or a PI3Kα inhibitor.

In certain embodiments, the pharmaceutical composition comprises one or more DUSP-MKP inhibitors that inhibit DUSP6 and/or DUSP1. In certain embodiments, the pharmaceutical composition comprises one or more DUSP-MKP inhibitors that decrease the activity of DUSP6 and/or DUSP1. In certain embodiments, the DUSP-MKP inhibitor is a DUSP6 inhibitor and inhibits DUSP6-induced dephosphorylation of extracellular signal-related kinase (ERK). In certain embodiments, the DUSP6 inhibitor sensitizes cancer cells to immune cell killing. In certain embodiments, the DUSP-MKP inhibitor is a DUSP1 inhibitor. In certain embodiments, the DUSP1 inhibitor sensitizes cancer cells to immune cell killing.

In certain embodiments, the presently disclosed subject matter provides for a pharmaceutical composition comprising an effective amount of a DUSP-MKP inhibitor that reduces cancer cell proliferation. In certain embodiments, the presently disclosed subject matter provides for a pharmaceutical composition comprising an effective amount of a DUSP-MKP inhibitor that promotes cancer cell death. In certain embodiments, the presently disclosed subject matter provides for a pharmaceutical composition comprising an effective amount of a DUSP-MKP inhibitor that inhibits cancer cell metastasis.

In certain embodiments, the presently disclosed subject matter provides for a pharmaceutical composition comprising an effective amount of an agent comprising a compound having the formula:

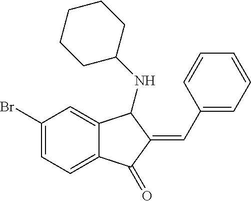 [SMILES: O=C1/C(=C/c2ccccc2)C(NC2CCCCC2)c2cc(Br)ccc21]

or a prodrug or an analog thereof, in an amount effective to increase levels of phosphorylated extracellular signal-related kinase (ERK) or to decrease levels of dephosphorylated ERK or in a cancer cell and sensitize the cancer cell to immune cell killing. In certain embodiments, the presently disclosed subject matter provides for a pharmaceutical composition comprising an effective amount of an agent that inhibits DUSP6-induced dephosphorylation of ERK and sensitizes cancer cells to immune cell killing. In certain embodiments, the presently disclosed subject matter provides for a pharmaceutical composition comprising an effective amount of a first agent that inhibits DUSP6-induced dephosphorylation of ERK and sensitizes cancer cells to immune cell killing and a second agent that promotes immune cell killing.

### 5.3 Methods of Treatment

The presently disclosed subject matter relates to methods of treating a cancer in which a dual specificity mitogen-activated protein kinase phosphatase (DUSP-MKP) inhibitor is used to sensitize cancer cells to immune cell killing.

In certain embodiments, the presently disclosed subject matter can be used to improve, increase, or enhance the anti-cancer response of a subject by administering, to the subject, a DUSP-MKP inhibitor that sensitizes cancer cells to immune cell killing, together with an agent that promotes immune cell killing of cancer cells.

In certain embodiments, the presently disclosed subject matter can be used to improve, increase, or enhance the anti-cancer response of a subject by administering, to the subject, a DUSP-MKP inhibitor that sensitizes cancer cells to immune cell killing, together with an agent that promotes immune cell killing of cancer cells by sensitizing cancer cells to lymphokine-activated killer (“LAK”) cell activity.

In certain embodiments, the subject has been determined to exhibit an inadequate anti-cancer response to checkpoint inhibitor therapy (“inhibitor monotherapy”) administered without a DUSP-MKP inhibitor), either by, for example, clinical history of the individual subject, by correlation to one or more biomarker, or by the cancer type involved. In certain non-limiting embodiments, treatment with the DUSP-MKP inhibitor can be instituted prior to treatment with an agent that promotes cell killing and the two types of therapy can or can not overlap in time. In certain embodiments, treatment with the DUSP-MKP inhibitor can be administered concurrently with the agent that promotes cell killing.

In certain embodiments, the presently disclosed subject matter provides for a method of treating cancer in a subject comprising administering, to the subject, (i) an amount of a dual specificity mitogen-activated protein kinase phosphatase (DUSP-MKP) inhibitor that sensitizes cancer cells to immune cell killing and (ii) an agent that promotes immune cell killing, for example, a cell-mediated anti-cancer immune response in the subject. In certain embodiments, the immune cell killing is immunogenic cell death (“ICD”). In certain embodiments, treatment with DUSP-inhibitor renders the cancer cells more sensitive to lymphokine-activated killer cell activity.

In certain embodiments, the presently disclosed subject matter provides for a method of treating cancer in a subject comprising administering, to the subject, (i) an amount of a dual specificity mitogen-activated protein kinase phosphatase (DUS-MKP) inhibitor that sensitizes cancer cells to immune cell killing and (ii) an agent that promotes immune cell killing, for example, a checkpoint inhibitor. In certain embodiments, the check point inhibitor is an antibody selected from the group consisting of an antibody for CTLA-4 (for example, ipilimumab), an antibody for PD-1 (for example, pembrolizumab, nivolumab, or BGB-A137), and an antibody for PD-L1 (for example, atezolizumab, avelumab, ordurvalumab). In certain non-limiting embodiments, the agent is an antibody for CD52 (for example, alemtuzumab), and an antibody for CD20 (for example, ofatumumab or rituximab).

In certain embodiments, the presently disclosed subject matter provides for a method of treating cancer in a subject comprising administering, to the subject, (i) an amount of a dual specificity mitogen-activated protein kinase phosphatase (DUSP-MKP) inhibitor that sensitizes cancer cells to immune cell killing and (ii) an agent that promotes immune cell killing, for example, natural killer cells and dendritic cells, wherein the immune cells are activated in vitro and introduced to the subject. Alternatively or additionally, the immune cells can comprise T cells or interleukin-2 (IL-2)-activated peripheral blood mononuclear cells (PBMCs). Said cells can be autologous or heterologous.

In certain embodiments, the presently disclosed subject matter provides for a method of treating cancer in a subject comprising administering, to the subject, (i) an amount of a dual specificity mitogen-activated protein kinase phosphatase (DUSP-MKP) inhibitor that sensitizes cancer cells to immune cell killing and (ii) an agent that promotes immune cell killing, for example, a lymphokine and/or a cytokine, for example, but not limited to, interleukin-2 and/or interferon alpha.

In certain embodiments, the presently disclosed subject matter provides for a method of treating cancer in a subject comprising:

(i) determining whether the subject expresses cancer cells that are resistant to treatment with an inhibitor monotherapy, wherein the resistant cells treated with the inhibitor monotherapy exhibit DUSP activity; and

(ii) where the subject expresses cancer cells that are resistant to treatment with the inhibitor monotherapy, treating the subject with a first agent comprising a DUSP-MKP inhibitor that sensitizes cancer cells to immune cell killing or a combination of the first agent comprising a DUSP-MKP inhibitor that sensitizes cancer cells to immune cell killing with a second agent that that promotes a cell-mediated anti-cancer immune response.

In certain embodiments, the presently disclosed subject matter provides for a method of treating cancer in a subject comprising administering to the subject in need thereof an effective amount of (i) a first agent that inhibits DUSP6-induced dephosphorylation of extracellular signal-related kinase (ERK) and sensitizes cancer cells to immune cell killing and (ii) a second agent that promotes immune cell killing.

In certain embodiments, the presently disclosed subject matter provides for a method for reducing cancer cell proliferation or promoting cancer cell death in a subject in need thereof comprising administering to the subject an effective amount of (i) a first agent comprising a DUSP-MKP inhibitor that sensitizes cancer cells to immune cell killing and (ii) a second agent that promotes immune cell killing.

In certain embodiments, the presently disclosed subject matter provides for a method for reducing cancer cell proliferation or promoting cancer cell death in a subject in need thereof comprising contacting a cancer cell of the subject with an effective amount of (i) a first agent comprising a DUSP-MKP inhibitor that sensitizes cancer cells to immune cell killing and (ii) a second agent that promotes immune cell killing.

In certain embodiments, the presently disclosed subject matter provides for a method for reducing cancer cell proliferation or promoting cancer cell death in a subject in need thereof comprising contacting a cancer cell of the subject with (i) a first agent comprising a compound having the formula:

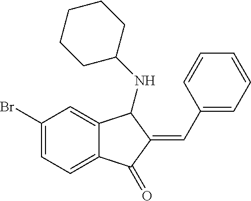 [SMILES: O=C1/C(=C/c2ccccc2)C(NC2CCCCC2)c2cc(Br)ccc21]

or an analog thereof, in an amount effective to increase levels of phosphorylated ERK or to decrease levels of de-phosphorylated ERK in the cancer cell and sensitize the cancer cell to immune cell killing and (ii) a second agent that promotes immune cell killing.

In certain embodiments, the presently disclosed subject matter provides for a method of inhibiting cancer cell metastasis in a subject in need thereof comprising administering to a subject in need thereof an effective amount of (i) a first agent comprising a dual specificity mitogen-activated protein kinase phosphatase (DUSP-MKP) inhibitor that sensitizes cancer cells to immune cell killing and (ii) a second agent that promotes immune cell killing.

In certain embodiments, the methods and formulations of the presently disclosed subject matter can be used for reducing, inhibiting, preventing or reversing cancer and/or tumor growth. The route of administration eventually chosen will depend upon a number of factors and can be ascertained by one skilled in the art.

In certain embodiments, cancers that can be treated according to the presently disclosed subject matter include but are not limited to breast cancer, cervical cancer, leukemia, for example, pre-B acute lymphoblastic leukemia pre-B ALL, prostate cancer, gastric cancer, pancreatic cancer, non-small cell lung carcinoma, and colon cancer, for example metastatic colon cancer.

In certain embodiments, the method of treating cancer in a subject comprises administering to the subject at least one of the disclosed pharmaceutical compositions that reduces and/or inhibits the activity and/or expression of a DUSP gene or one or more of downstream targets of the DUSP gene.

In certain embodiments, the method of treating cancer in a subject comprises determining whether the subject is resistant to inhibitor monotherapy. In certain embodiments, the method of treating cancer in a subject comprises determining whether the cancer comprises cells that are resistant to inhibitor monotherapy.

In certain embodiments, a method of treating a subject can comprise determining if the subject and/or the cancer cells of the subject are resistant to an inhibitor monotherapy and/or a cancer monotherapy, where if the subject and/or the cancer cells of the subject are resistant to an inhibitor monotherapy and/or a cancer monotherapy, then treating the subject with a therapeutically effective amount of a DUSP-MKP inhibitor or a therapeutically effective amount of a DUSP-MKP inhibitor and an agent that promotes a cell-mediated anti-cancer immune response.

The presently disclosed subject matter relates to the administration of a DUSP-MKP inhibitor for the treatment of a cancer, and also to the administration of a DUSP-MKP inhibitor in combination with an anti-cancer agent and/or a cancer therapy and/or immunotherapy agent for the treatment of a cancer.

An anti-cancer agent can be any molecule, compound chemical or composition that has an anti-cancer effect. Anti-cancer agents include, but are not limited to, chemotherapeutic agents, radiotherapeutic agents, cytokines, anti-angiogenic agents, apoptosis-inducing agents or anti-cancer immunotoxins. In certain non-limiting embodiments, an inhibitor can be administered in combination with one or more anti-cancer agents.

In certain embodiments, the cancer therapy comprises cryotherapy, radiation therapy, chemotherapy, hormone therapy, biologic therapy, bisphosphonate therapy, high-intensity focused ultrasound, frequent monitoring, frequent prostate-specific antigen (PSA) checks and radical prostatectomy. A non-limiting example of a biologic therapeutic is Sipuleucel-T. Bisphosphonate therapy includes, but is not limited to, clodronate or zoledronate. In certain embodiments, these methods can be used to produce an anti-cancer effect in a subject.

Hormone therapy can include one or more of orchiectomy and the administration of luteinizing hormone-releasing hormone (LHRH) analogs and/or agonists, LHRH antagonists, anti-androgens or androgen-suppressing drugs. Non-limiting examples of LHRH analogs and/or agonists include leuprolide, goserelin and buserelin. Non-limiting examples of LHRH antagonists include abarelix, cetrorelix, ganirelix and degarelix. Anti-androgen drugs include, but are not limited to, flutamide, bicalutamide, enzalutamide and nilutamide. Non-limiting examples of androgen-suppressing drugs include estrogens, ketoconazole and aminoglutethimide. Frequent monitoring can include PSA blood tests, digital rectal exams, ultrasounds and/or transrectal ultrasound-guided prostate biopsies at regular intervals, e.g., at about 3 to about 6 month intervals, to monitor the status of the prostate cancer. Radical prostatectomy is a surgical procedure that involves the removal of the entire prostate gland and some surrounding tissue. Prostatectomies can be performed by open surgery or it may be performed by laparoscopic surgery.

“In combination with,” as used herein, means that the inhibitor and the one or more anti-cancer agents are administered to a subject as part of a treatment regimen or plan. This term does not require that the inhibitor and/or DUSP-MKP inhibitor and one or more anti-cancer agents are physically combined prior to administration nor that they be administered over the same time frame.

### 5.4 Kits

The presently disclosed subject matter further provides kits that can be used to practice the presently disclosed embodiments. For example, and not by way of limitation, a kit of the present invention can comprise a DUSP-MKP inhibitor or a pharmaceutical formulation comprising a therapeutically effective amount of a DUSP-MKP inhibitor. In certain embodiments, a kit of the presently disclosed subject matter can comprise first agent comprising a DUSP-MKP inhibitor that sensitizes cancer cells to immune cell killing and can further comprise a second agent that promotes immune cell killing, e.g., within the same container as the pharmaceutical composition comprising a DUSP-MKP inhibitor (or formulation thereof) or within a second container.

In certain embodiments, the second agent can be an agent that promotes a cell-mediated anti-cancer immune response in the subject. In certain embodiments, the second agent can be immune cells selected from the group consisting of natural killer cells and dendritic cells, wherein the immune cells are activated in vitro and introduced to the subject. In certain embodiments, the immune cells can comprise T cells or interleukin-2 (IL-2)-activated peripheral blood mononuclear cells (PBMCs). In certain embodiments, the immune cells can be autologous. In certain embodiments, the cells can be heterologous. In certain embodiments, the second agent that promotes immune cell killing can be a lymphokine, for example but not limited to interleukin-2 or interferon alpha. In certain non-limiting embodiments, the second agent is a PI3Kα inhibitor.

In certain embodiments, the second agent sensitizes cancer cells to lymphokine-activated killer (“LAK”) cell activity. In certain embodiments, the agent that promotes immune cell killing of cancer cells is a checkpoint inhibitor. In certain embodiments, a checkpoint inhibitor is an antibody selected from the group consisting of an antibody for CTLA-4 (for example, ipilimumab), an antibody for PD-1 (for example, pembrolizumab, nivolumab, or BGB-A137), and an antibody for PD-L1 (for example, atezolizumab, avelumab, ordurvalumab). In certain embodiments, the agent is an antibody for CD52 (for example, alemtuzumab), and an antibody for CD20 (for example, ofatumumab or rituximab).

In certain non-limiting embodiments, the present invention provides for a kit for use in treating cancer in a subject comprising a DUSP-MKP inhibitor or a pharmaceutical formulation thereof, a second agent and instructions for use. For example, and not by way of limitation, the instructions can indicate that the DUSP-MKP inhibitor and the second agent can be administered together or separately. In certain embodiments, the kit is for use in treating breast cancer, cervical cancer, leukemia, for example, pre-B acute lymphoblastic leukemia pre-B ALL, prostate cancer, gastric cancer, pancreatic cancer, non-small cell lung carcinoma, and colon cancer, for example metastatic colon cancer.

In certain non-limiting embodiments, the present invention provides for a kit that includes a vial comprising a pharmaceutical composition comprising a DUSP-MKP inhibitor, e.g., a therapeutically effective amount of a DUSP-MKP inhibitor, and/or a vial comprising a second agent, e.g., a therapeutically effective amount of a second agent, with instructions to use any combination of the components of the one or more vials together or separately for treating cancer. For example, and not by way of limitation, the instructions can include a description of a DUSP-MKP inhibitor and/or a second agent, and, optionally, other components present in the kit. In certain embodiments, the instructions can describe methods for administration of the components of the kit, including methods for determining the proper state of the subject, the proper dosage amount and the proper administration method for administering a DUSP-MKP inhibitor and/or a second agent. Instructions can also include guidance for monitoring the subject over the duration of the treatment time. In certain embodiments, the kit can further comprise one or more vials comprising additional DUSP-MKP inhibitors and/or other agents. In certain embodiments, a kit of the present invention comprises a vial that includes a DUSP-MKP inhibitor and the second agent.

In certain non-limiting embodiments, the present invention provides for a kit of this disclosure further including one or more of the following: devices and additional reagents, and components, such as tubes, containers, cartridges, and syringes for performing the methods presently disclosed.

In certain embodiments, the presently disclosed subject matter provides for a kit comprising:
(i) one or more agent that can (a) decrease/inhibit the activity of DUSP6; (b) decrease the activity DUSP6 and DUSP1; (c) sensitize cancer cells to immune cell killing; and (d) reduce or inhibit cancer cell and/or tumor cell growth; and
(ii) one or more agent that can promote immune cell killing.

In certain embodiments, the presently disclosed subject matter provides for a kit comprising a container comprising:
(i) an effective amount of a first agent comprising a DUSP-MKP inhibitor comprising BCI-215 or an analog thereof that sensitizes cancer cells to immune cell killing;
(ii) an effective amount of a second agent that promotes immune cell killing; and
(iii) a pharmaceutically acceptable buffer.

The following example is offered to more fully illustrate the disclosure, but is not to be construed as limiting the scope thereof.

### 6. EXAMPLE

### 6.1 Methods

Compounds and Chemicals.

BCI-215 (12) was described previously. Sanguinarine, menadione, NSC95397, BCI, JNK-IN-8, doxorubicin, and cisplatin were obtained from Sigma-Aldrich. CellTracker™ Green (Molecular Probes C2925), chloromethyl fluorescein diacetate, acetyl ester (CM-H2-DCFDA, Molecular Probes C6827), Tetramethylrhodamine, ethyl ester (TMRE, Molecular Probes T-669), and dihydroethidium (DHE, Molecular Probes D1168) were obtained from ThermoFisher. Other MAPK inhibitors were obtained from Selleckchem (SCH772984, cat#57101; SP600125, cat#S1460; SB203580, cat#S1076). Ficoll-Paque was obtained from GE Healthcare Life Sciences. Interleukin 2 was a generous gift of Prometheus, Inc. The Annexin V/PI Apoptosis Detection Kit FITC was from eBioscience.

Hepatocyte Mitochondrial Function.

Rat hepatocytes were isolated using standard two step collagenase digestion (15) and sub-cultivated at 14,000 hepatocytes/well in collagen-1 coated 384 well plates in Williams E Medium supplemented with 10% FBS, 2 mM L-glutamine and 50 U/ml Penicillin and streptomycin. After 4 hours, medium was decanted and replaced with Hepatocyte Maintenance Media (Williams E supplemented with 1.25 mg/ml bovine serum albumin, 6.25 μg/ml human insulin, 100 nM dexamethasone, 6.25 μg/ml human transferrin, 6.25 ng/ml selenous acid, 2 mM L-glutamine, 15 mM HEPES, 100 U/mL penicillin, and 100 U/mL streptomycin). After overnight culture, cells were treated with concentration gradients of test agents. One hour after compound addition, cells were labeled with 200 nM TMRE and 4 μg/ml Hoechst 33342 for 45 min, imaged live on an ArrayScan VTI using a 10× objective, and images were analyzed with the Compartmental Bioapplication. Mitochondria were identified as cytosolic spots by size and brightness. The final parameter was % HIGH RingSpotAvgIntenCh2 (i.e., percentage of cells with TMRE puncta in the cytoplasm based on a threshold set with vehicle treated cells).

ROS Generation in Hepatocytes.

Cells were cultured as above and labeled four hours following drug addition with 4 μM DHE for 2 hours. Hoechst 33342 was added to a 4 μg/ml final concentration during the final hour of incubation. In the presence of ROS, DHE is oxidized to a red fluorescent dye (oxyethidium). Cells were imaged as above and the percentage of oxyethidium-nuclear positive cells calculated based on a threshold set with vehicle treated cells.

Five-Day Hepatocyte Toxicity.

A gelling solution of 1.25 mg/ml rat tail collagen type I in pH 7.2 90:10 (v/v) Williams E media/10×HBSS was overlaid onto the rat hepatocytes. The collagen gel was incubated for 1 hour at 37° C., 5% CO2. The collagen sandwich cultures were then challenged for 5 days to test compounds in Hepatocyte Maintenance Media, without refeeding. A 10× solution of 40 μg/ml Hoechst 33342 was added during the final 2 hours of incubation followed by a 10× solution of 20 μg/ml PI for 1 hour. Cells were imaged and the percentage of PI positive cells calculated as above.

Zebrafish.

All procedures involving zebrafish were reviewed and approved by the University of Pittsburgh Institutional Animal Care and Use Committee. Wildtype AB* embryos were kept in E3 medium (5 mM NaCl, 0.17 mM KCl, 0.33 mM CaCl₂, 0.33 mM MgSO₄). At 48 hours post fertilization (hpf), embryos were arrayed into the wells of a 96 well microplate and treated with vehicle (0.5% DMSO) or test agents. After a 30-min pre-incubation, embryos were labeled with a solution of 10 μM DHE and 40 μg/mL MS222 (tricaine methanesulfonate, Sigma) in E3 to restrict movement during imaging. Six hours after DHE loading, embryos were imaged on an ArrayScan II using a 2.5× objective. Images were analyzed for oxyethidium fluorescence with the TargetActivation Bioapplication using the MEAN_ObjectAvgIntenCh1 parameter.

Cell Culture.

MDA-MB-231 and BT-20 breast cancer and HeLa cervical cancer cell lines were obtained in 1997, 2013, and 2000, respectively, from the American Culture Type Collection (ATCC, Manassas, Va.) and maintained as recommended. MDA-MB-231 and HeLa cells were re-authenticated in 2011 by The Research Animal Diagnostic Laboratory (RADIL) at the University of Missouri, Columbia, Mo. using a PCR based method that detects 9 short tandem repeat (STR) loci, followed by comparison of results to the ATCC STR database. Original ATCC stocks and re-authenticated cells were cryopreserved in liquid nitrogen and maintained in culture for no more than ten passages or three months, whichever was shorter, after which cells were discarded and a new vial thawed.

HCA of Apoptosis and ERK Phosphorylation.

MDA-MB-231 cells (10,000/well) were treated with identical concentration gradients of test agents on the right and left half of a 384 well microplate for later assessment of potential compound autofluorescence. After 24 hours, cells were fixed, permeabilized with 0.2% Triton X-100, blocked with 1% BSA/PBS, and immunostained with anti-phospho-ERK (E10, CST cat#9106L)/AlexaFluor488 and anti-cleaved caspase-3 (CST cat#9664L)/AlexaFluor594 primary/secondary antibody pairs. Plates were imaged on the ArrayScan II using a 10×0.5NA objective. Each well was background corrected by subtracting mean phospho-ERK and cleaved caspase-3 intensities from wells that had received secondary antibody only. Data in Table 1 are the averages of the indicated numbers of independent experiments, each performed in quadruplicate. IC50, standard error, and 95% confidence intervals were calculated by two-way ANOVA with Bonferroni correction in GraphPad Prism.

Detection and Quantitation of ROS in Cancer Cells.

Detection and quantitation of ROS in cancer cells was performed as described before (14). Briefly, MDA-MB-231 cells were labeled with Hoechst 33342, loaded with CM-H2-DCFDA (5 μM, 15 min), and treated with test agents for 10 min. After two washes, cells were analyzed for Hoechst and ROS/FITC fluorescence on the ArrayScan II. Cells were classified as positive for ROS if their average FITC intensity exceeded a threshold defined as the average FITC intensity plus one SD from DMSO-treated wells.

HCA of Cell Motility and Cytotoxicity.

HCA of cell motility and cytotoxicity was performed essentially as described (51). MDA-MB-231 cells (15,000/well) were plated in collagen-coated Oris™ Pro 384-well microplates (Platypus Technologies cat # PRO384CMACCS) containing a chemical exclusion zone that dissolves upon cell seeding. Two hours after plating, medium was removed, and cells treated with ten-point, two-fold concentration gradients of test agents. Forty-eight hours after treatment, cells were stained with 10 μg/ml Hoechst 33342 and 1 μg/ml PI in HBSS for 15 min at 37° C. Plates were washed once with PBS and scanned live on the ArrayScan II using a 5× objective. To capture cells that had entered into the exclusion zone, a single field was acquired in the center of the well and nuclei therein enumerated. To assess changes in cell loss, nuclear size, and necrotic cell death, a second scan was performed that captured one field at the edge of the well (51). Parameters exported and plotted were SelectedObjectCountPerValidField (cell density), MEAN ObjectAreaCh1 (nucleus size), and % RESPONDER MeanAvgIntenCh2 (percent PI positive cells based on based on a threshold set with vehicle treated cells).

Colony Formation in Three Dimensional Matrigel Culture.

MDA-MB-231 cells (2000/well) transduced with a biosensor consisting of EGFP with a mitochondrial targeting sequence derived from cytochrome-C oxidase subunit VIII (52) were trypsinized, resuspended in RPMI1640 containing 2% FBS and 2% matrigel, and seeded in 384 well microplates on a 15 μl cushion of undiluted matrigel. After 24 hours, cells were treated with various concentrations of BCI-215 or vehicle (0.2% DMSO). After two days, drug was washed out and cells allowed to expand for an additional three to five days. At the end of the study, medium was replaced with HBSS containing 4 μg/ml PI for 1 hour, and plates scanned live on an ImageXpress Ultra HCS reader, acquiring z-stacks (4× objective, 20 planes, 50 μm) in the green and red channels. Cell numbers were quantified from maximum projection images using the Multiwavelength Cell Scoring application.

Western Blotting.

Western blotting was performed as described before (14). Antibodies were: pERK (T202/Y204, CST9101), total ERK (CST9102), pJNK, (T183/Y185, CST9251), total JNK, (CST9252), pp38 (T180/Y182, CST9215), total p38 (CST9212), (pMEK1/2 (S217/221, CST9121), total MEK1/2 (CST9122), pMKK4/SEK1 (S257, CST4514), MKK4/SEK1 (CST3346), GAPDH (abeam 8245). Antibodies were used at 1:1000 dilution except pJNK (1:500) and GAPDH (1:2000).

Toxicity Reversal.

Cells were pre-treated (30 min for SCH772984, SP600125, SB203580, and 3 hours for JNK-IN-8) with identical concentration gradients of MAPK inhibitors on the right and left halves of a 384 well microplate. After preincubation, half of the microplate was treated with vehicle (DMSO), the other with a pro-apoptotic concentration of BCI-215 (25 μM). To eliminate potential bias through plate/edge effects, an independent plate was prepared in parallel where vehicle and BCI-215 treatments were reversed. Twenty-four hours thereafter, plates were stained with Hoechst 33342, washed once, and imaged on the ArrayScan II using a 10× objective for analysis for cell numbers and nuclear morphology. Plates were subsequently immunostained with a cleaved caspase-3/Cy5-conjugated secondary antibody pair and analyzed for apoptosis on an ArrayScan VTI using a 20×0.75 NA objective. Four independent readouts were extracted and correlated: cell density (SelectedObjectCountPerValidField), nuclear condensation (MEAN_ObjectAvgIntenCh1), nucleus rounding (MEAN_ObjectShapeLWRCh1), and average cellular cleaved caspase-3 intensity (MEAN_AvgIntenCh2). For each parameter, data were normalized to vehicle (maximum rescue) and BCI-215 (no rescue) as % rescue=1−((data point−DMSO)/(DMSO-BCI-215))*100.

Immune Cell Killing.

Peripheral blood mononuclear cells were obtained from healthy volunteers with an established IRB approved protocol, separated from heparinized blood on Ficoll-hypaque (GE Healthcare, Chicago) gradients as previously reported (16). Cells were cultured in RPMI 1640 supplemented with 10% FCS, 1% glutamine, 1% penicillin/streptomycin, and stimulated with 6,000 IU of Interleukin 2 for 24 hours. After incubation, cells were washed with PBS and counted. In parallel, MBA-MB-231 cells were pre-treated in a 384 well plate with vehicle or BCI-215 (3 μM). After 24 hours in culture, medium was replaced and PBMC added in two-fold serial dilutions starting with a 50-fold excess of PBMCs in triplicate. After 24 hours of co-culture, cells were fixed with formaldehyde/Hoechst 33342, washed twice with PBS, and imaged on the ArrayScan II. Cancer cells were identified by their larger nuclei compared with PBMC, setting a size gate in the DAPI channel. In experiments with chemotherapeutics, cells carrying a biosensor consisting of a mitochondrial targeting sequence derived from cytochrome c oxidase VIII linked to GFP that is a surrogate for cytochrome c release from mitochondria (17) were pre-treated for 24 hours with cisplatin (2 μM) or doxorubicin (400 nM), exposed to LAK as above, and cancer cells identified and quantified by green fluorescence. Cell densities were normalized to those in the absence of PBMCs. Mean cell densities from multiple independent experiments were averaged and plotted in GraphPad Prism.

Flow Cytometry.

Flow cytometric analysis was performed on a C6 flow cytometer (Accuri Cytometers, Ann Arbor, Mich., USA) instrument within the University of Pittsburgh Cancer Institute Flow and Imaging Cytometry core facility and analyzed using FlowJo software (Tree Star Inc, Ashland, Oreg., USA). Single cell suspensions were stained with Annexin/PI (eBioscience) according to the manufacturer's protocol. Cells were identified via forward and side scatter and gated accordingly. All assessments were performed immediately after 30 min of incubation at 37° C. Necrotic, early, and late apoptotic cells were defined as cells that stained positive for PI only, annexin V only, or PI and annexin V, respectively.

Statistical Analysis.

Multiple data points were analyzed in GraphPad Prism by one-way ANOVA using Dunnett's multiple comparison test. EC50/IC50 values were obtained from at least three independent experiments by non-linear regression using a four parameter logistic equation. IC50, standard error, and 95% confidence intervals were calculated in GraphPad.

### 6.2 RESULTS

BCI-215 Lacks Oxidative Toxicity to Rat Hepatocytes.

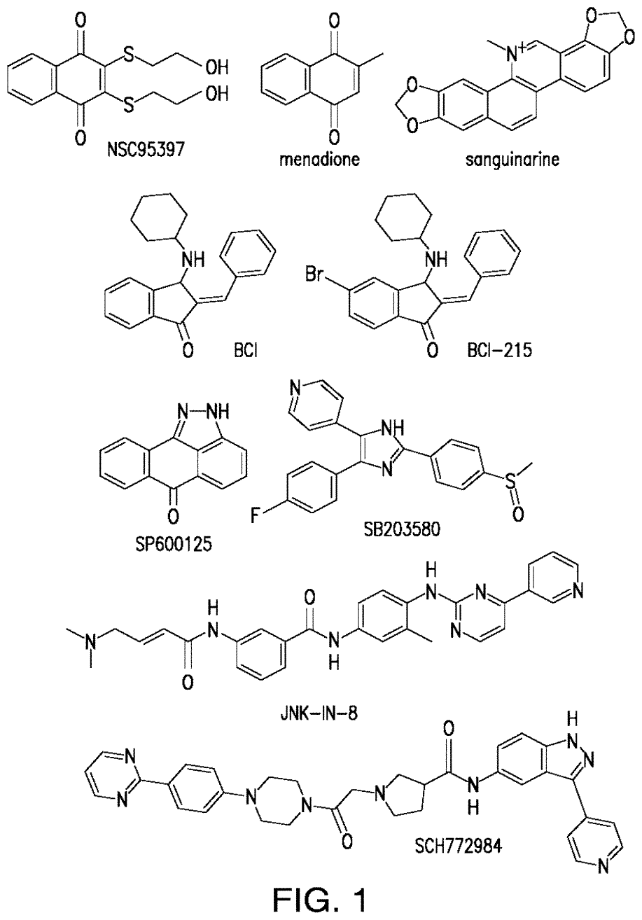

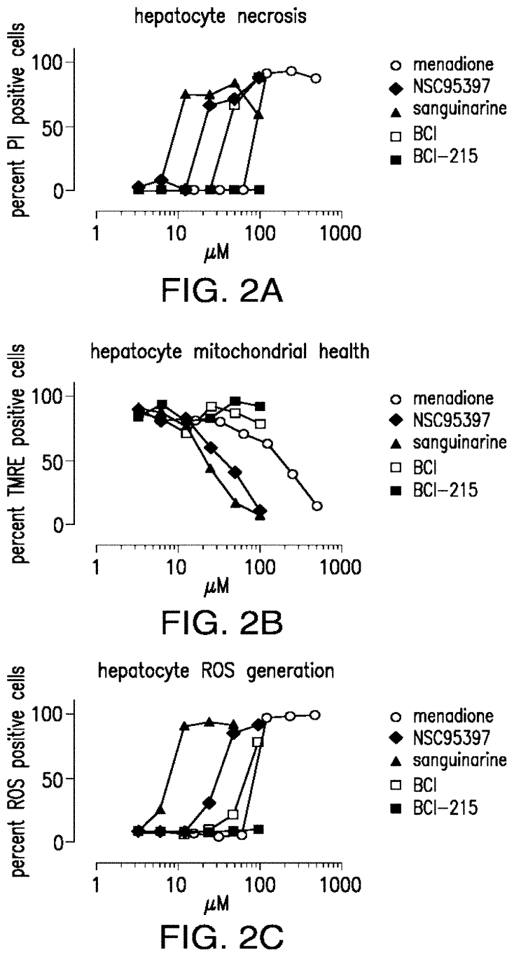

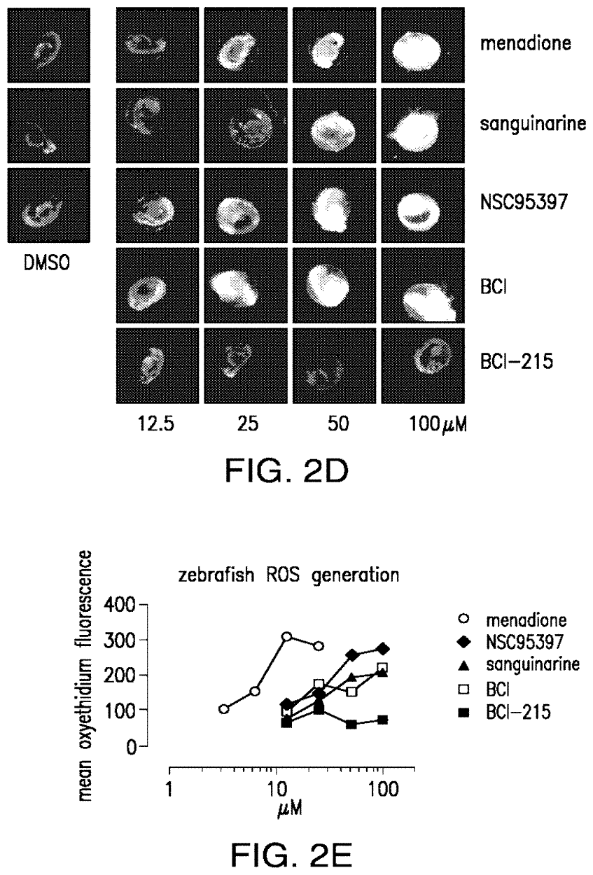

Previous studies of developmental toxicity were extended to a clinically relevant cell type. Freshly isolated rat hepatocytes were plated into 96 well plates and treated with two-fold concentration gradients of BCI-215, three previously described DUSP inhibitors (sanguinarine (13), NSC95397 (14), BCI (11), and menadione as a positive control for hepatotoxicity (FIG. 1). Toxicity was assessed by a live cell, high-content assay counting propidium iodide (PI) positive cells after a 5-day exposure, and through tetramethylrhodamine ethyl ester (TMRE) staining of mitochondria, which predicts hepatotoxicity due to mitochondrial damage in the clinic with high concordance (18). NSC95397, sanguinarine, and menadione caused cell death that correlated with loss of mitochondrial membrane integrity (FIG. 2A, 2B). BCI caused cell death but did not affect mitochondrial potential. BCI-215 was completely devoid of hepatocyte toxicity up to 100 showing low hepatic toxicity if developed into a potential therapeutic.

BCI-215 does not Generate Reactive Oxygen Species (ROS) in Hepatocytes or in Developing Zebrafish Larvae.

Generation of ROS by dihydroethidium (DHE) staining was quantified. Like mitochondrial membrane potential, ROS generation is one of the best predictors of clinical hepatotoxicity (18). From a mechanistic perspective, compounds that generate ROS can lead to non-specific, irreversible inactivation of PTPs and DUSPs. The active site of all PTPs and DUSPs contains a nucleophilic cysteine that is extremely sensitive to oxidation, and while mild, reversible oxidation is a physiological mechanism to regulate activity (19), oxidation past the sulfinic acid stage is irreversible (20). Irreversible oxidation is expected for the naphthoquinone NSC95397, which generates ROS in MDA-MB-231 breast cancer cells (14), and sanguinarine, which depletes glutathione levels (21). With the exception of BCI-215, all agents generated ROS in hepatocytes (FIG. 2C), providing both a mechanism for BCI-215's lack of toxicity and eliminating the possibility of nonselective phosphatase inactivation. All agents that caused ROS in hepatocytes also caused ROS in zebrafish embryos, although their IC50 values were slightly different in the two models, possibly reflecting differences in compound uptake or metabolism (FIG. 2D, 2E). These findings document that the cellular activities of BCI-215 in zebrafish are not mediated by nonselective oxidative processes.

BCI-215 has Antimigratory and Pro-Apoptotic Activities in Breast Cancer Cells that Correlate with Induction of ERK Phosphorylation.

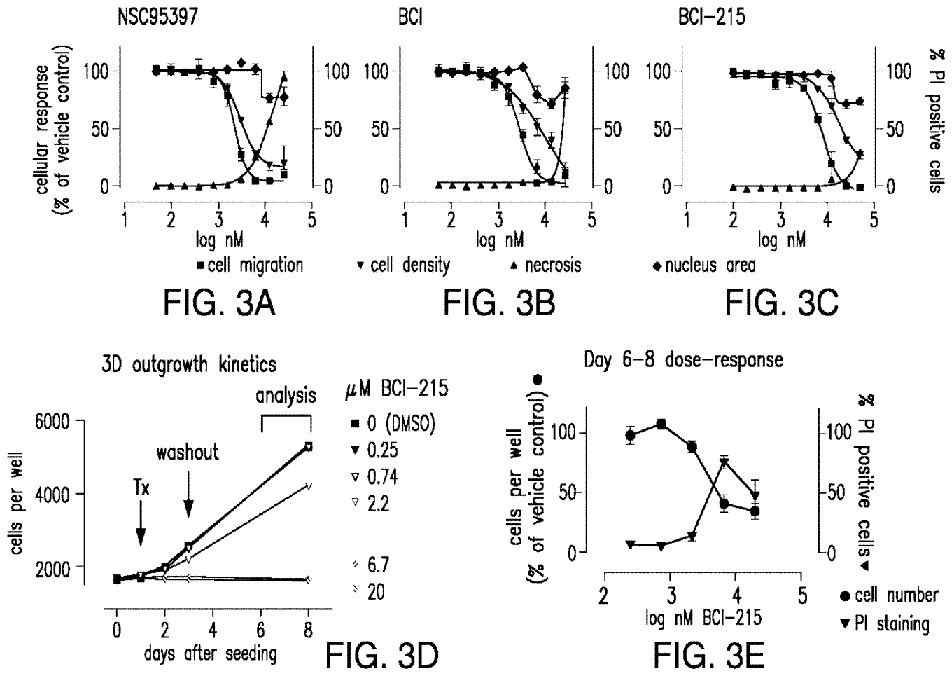

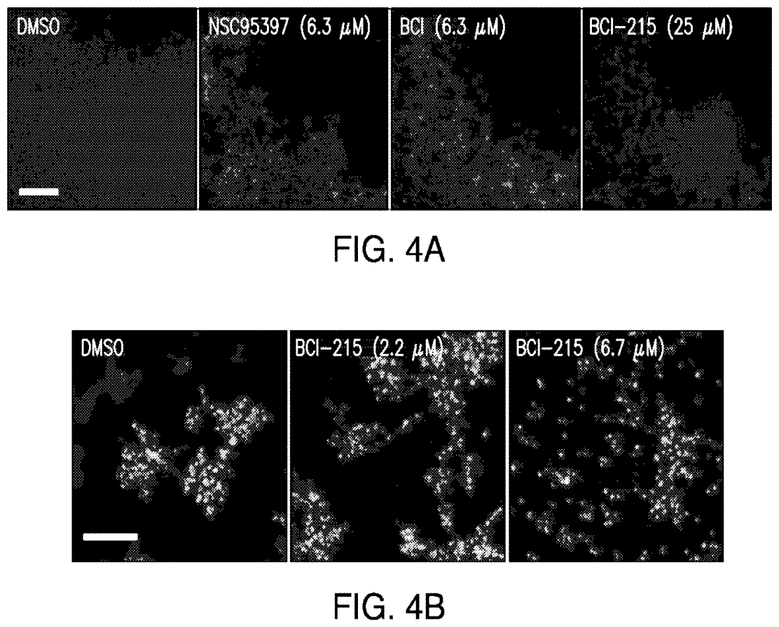

To investigate whether BCI-215 was toxic to cancer cells, MDA-MB-231 cells were plated in an Oris™ Pro 384 cell migration plate and treated with ten-point concentration gradients of NSC95397, BCI, or BCI-215. Forty-eight hours following treatment, cells were stained live with PI and Hoechst 33342, and the percentage of PI positive cells was quantified on an ArrayScan II (ThermoFisher, Pittsburgh) high-content reader. All agents inhibited cell motility and attachment, and showed nuclear shrinkage with IC50 values between 7 and 15 μM (FIG. 3). NSC95397 is a chemically reactive structure and caused necrosis at antimigratory concentrations (FIG. 3A, % PI positive cells). Necrosis was reduced with BCI, and BCI-215 showed no signs of necrosis at antimigratory or pro-apoptotic concentrations (FIGS. 3B, 3C and 4A). BCI-215 also inhibited colony formation in the “matrigel-on-top” model, where cells are seeded at low densities, recapitulating an initial dormancy-like state followed by clonal outgrowth (22). MDA-MB-231 cells were transduced with a mitochondrial-targeted, GFP-labeled cytochrome C biosensor (17) to enable continuous live monitoring of colony growth, plated on a layer of matrigel and treated 24 hours later with various concentrations of BCI-215. Following two days of exposure, the drug was removed and cells were allowed to expand for an additional 4-6 days. At the end of the study, the cells were incubated with PI and analyzed for cell numbers and PI positivity by high content analysis (HCA). In contrast to the short term 2D assay, BCI-215 treated cells showed pronounced cell lysis in the longer-term 3D matrigel assay (FIGS. 3D, 3E and 4B).

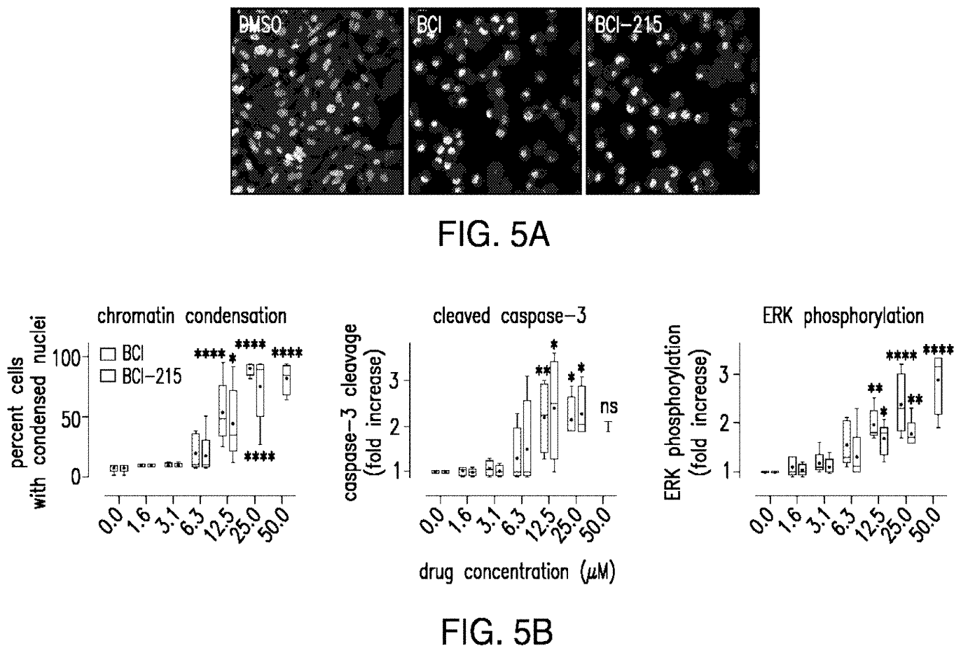

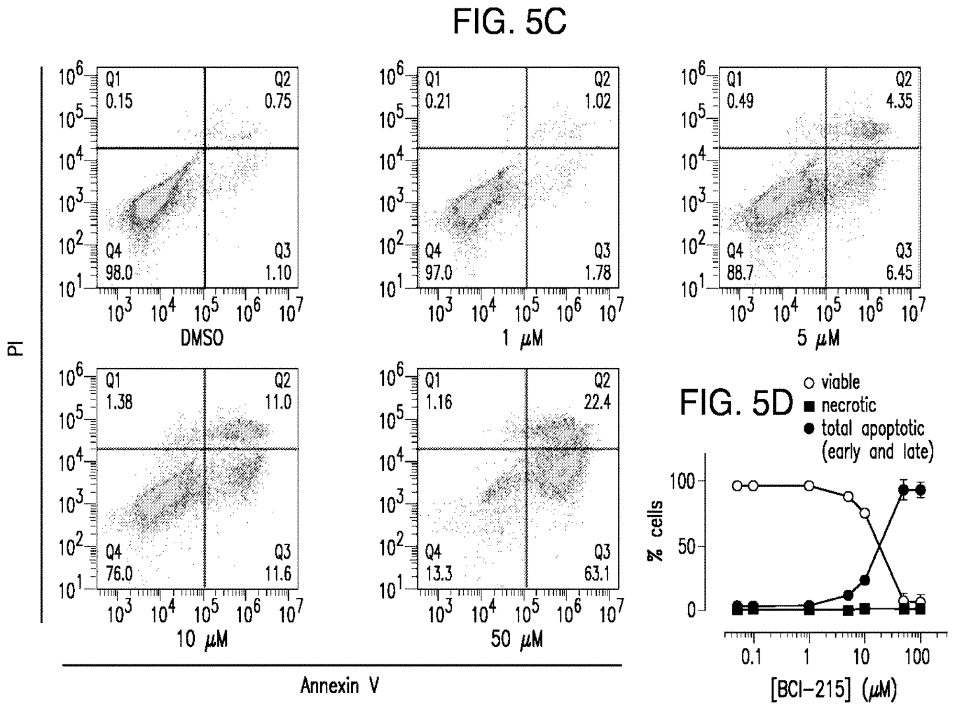

To probe mechanisms of BCI-215 induced cell death, multiplexed HCA of nuclear morphology was performed, caspase-3 cleavage (apoptosis) and ERK phosphorylation as a pharmacodynamic biomarker for DUSP-MKP inhibition. FIG. 5A shows that BCI and BCI-215 produced shrunken, condensed nuclei that resembled pyknosis, an early apoptotic event (23). Simultaneous quantitation of condensed nuclei, caspase-3 cleavage, and ERK phosphorylation revealed that both agents caused apoptosis that correlated with ERK phosphorylation (FIG. 2B). Based on their IC50 values, BCI and BCI-215 were equipotent (Table 1); at the highest concentration tested (50 however, BCI's non-specific toxicity impaired specific cellular measurements. Flow cytometric analysis confirmed apoptotic death and documented that PI positivity was a result of secondary cell membrane permeability, occurring only in Annexin V positive cells.

TABLE 1
Quantification of multiparametric evaluation of cellular
toxicity, caspase-3 activation, and ERK phosphorylation.

| | | IC50 | | | |
|---|---|---|---|---|---|
| Compound | Parameter | (μM) | SE | 95% CI | n |
| BCI | Nuclear condensation | 12.85 | 1.24 | 8.261 to 20.00 | 5 |
| BCI-215 | Nuclear condensation | 12.77 | 1.21 | 8.633 to 18.89 | 5 |
| BCI | ERK phosphorylation | 8.59 | 1.18 | 6.137 to 12.03 | 5 |
| BCI-215 | ERK phosphorylation | 15.37 | 1.21 | 10.35 to 22.81 | 5 |
| BCI | Caspase-3 cleavage | 9.17 | 1.22 | 6.019 to 13.97 | 4 |
| BCI-215 | Caspase-3 cleavage | 7.33 | 1.25 | 4.609 to 11.66 | 4 |

BCI-215 Sensitizes Cancer Cells to Immune Cell Killing.

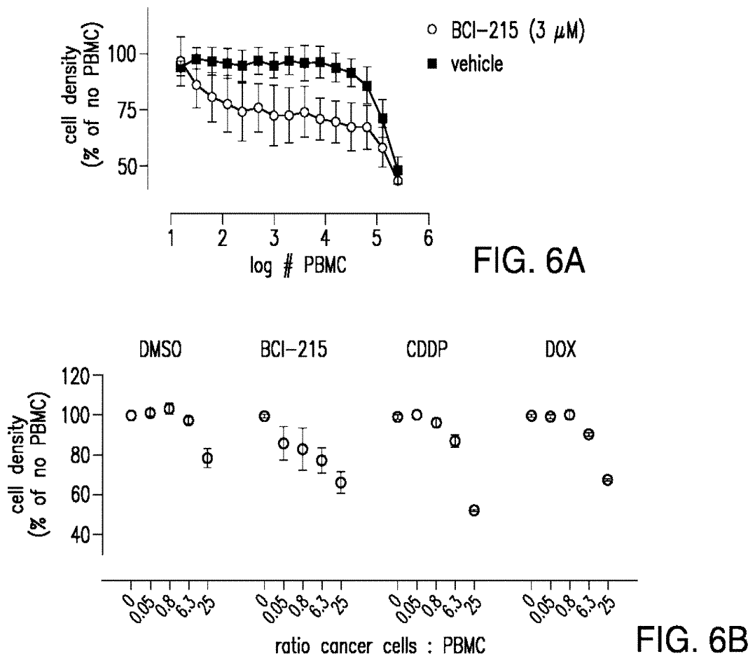

Immune system-targeted therapies are perhaps the greatest advance in cancer treatment in the last 50 years. Despite the spectacular success with immune checkpoint inhibitors, the majority of patients do not respond (24). Thus, there is an urgent need to develop effective therapies for those patients that do not achieve durable responses, and other mechanisms of resistance should be considered including the “lymphoplegic” effects of damage associated molecular pattern (DAMP) molecule release (25). A promising approach to harness the immune system in the response to small molecules is immunogenic cell death (ICD) (26). In ICD, tumor cells undergoing apoptosis display and secrete factors that recruit immune cells to the tumor bed and enhance cell killing activity. To test whether BCI-215 can sensitize cancer cells to immune cell kill, MDA-MB-231 cells were treated with vehicle or a mildly toxic concentration of BCI-215 (3 μM) for 24 hours followed by addition of interleukin-2 (IL-2)-activated peripheral blood mononuclear cells (PBMC). After an additional 24-hour incubation, cells were fixed, stained with Hoechst 33342, and imaged on the ArrayScan II. Cancer cell nuclei were gated by their larger size compared with PBMC. FIG. 6A shows dose-response curves of activated PBMC added to cells pre-treated with vehicle or BCI-215, averaged from three separate experiments. In the presence of vehicle alone, cells were relatively insensitive to immune cell kill; a maximal effect was obtained with a 20-fold excess of LAK; the EC50 was about a 10-fold excess (50,000 LAK/well). In the presence of BCI-215, the kill curve was shifted dramatically to lower numbers of PBMC, with maximal sensitization seen with as few as 1000 LAK/well, and EC50s of as few as 100 LAK/well, well over three log differences in killing. The effects of BCI-215 are then compared to two clinically used chemotherapeutic agents, doxorubicin and cisplatin, which have previously been reported to increase LAK activity in cell culture (27, 28). All agents sensitized cells to LAK activity; however, BCI-215 consistently showed sensitization at lower effector ratios than cisplatin or doxorubicin (FIG. 6B).

BCI-215 Induces Mitogenic and Stress Signaling in Cancer Cells without Generating ROS.

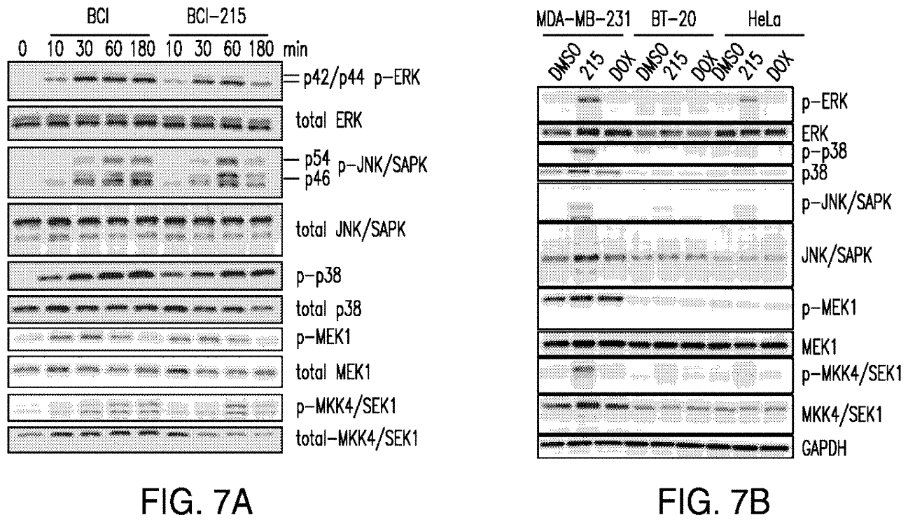

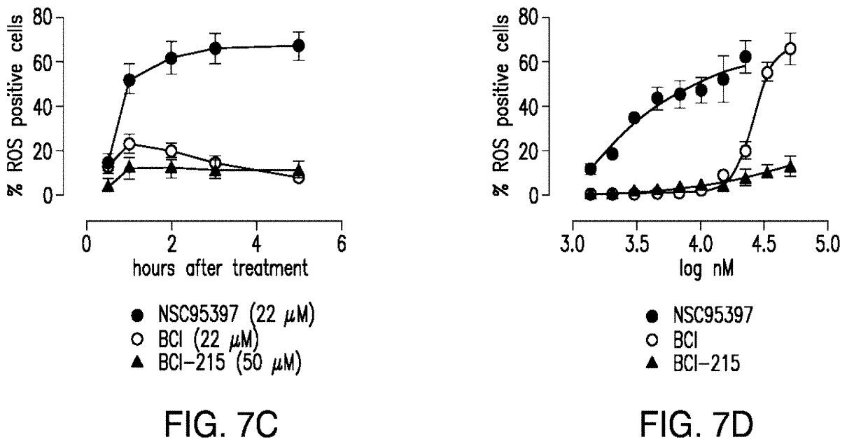

DUSP-MKPs have unique but overlapping substrate specificities. For example, DUSP6/MKP-3 is specific for ERK, whereas DUSP1/MKP1 dephosphorylates ERK, JNK/SAPK, and p38 (2). To establish a MAPK pathway activation profile and to corroborate the results from the immunofluorescence analysis, Western blot analysis of the kinetics of p-ERK, p-JNK/SAPK, and p-p38 induction in MDA-MB-231 cells at cytotoxic concentrations of BCI and BCI-215 (20 μM) was performed. FIG. 7A shows that both agents activated all three kinases with identical kinetics. Similar activation of signaling pathways was observed in a second TNBC line with different mutational profile and morphology (BT-20) and a non-breast cancer line (HeLa). Doxorubicin was included as a negative control that requires several hours for MAPK activation because of transcriptional downregulation of DUSP1/MKP-1 (29).

BCI-215 also activated MEK1 and MKK4/SEK1, which are upstream of ERK (30) and p38/JNK, respectively (31) (FIG. 7B). While MEK1 activation was minor and cell type dependent, MKK4/SEK1 was activated in all three lines (FIG. 7B), showing that BCI-215 can induce a general stress response. Because stress responses are usually accompanied by ROS generation (32), MDA-MB-231 cells were analyzed for generation of ROS in the presence of DUSP-MKP inhibitors. Cells were pre-labeled for 15 min with dichloromethyl-fluorescein diacetate, acetyl ester (CM-H2-DCFDA) and treated with various concentrations of NSC95397, BCI, or BCI-215. At various time points, cells were imaged live on the ArrayScan II. The para-quinone NSC95397 generated ROS within 30 min, with an EC50 of about 3-5 μM (FIG. 7C). This response was diminished with BCI (EC50: 20 μM). BCI-215, at 50 μM (more than 5× the EC50 for apoptosis and p-ERK induction), did not generate ROS in MDA-MB-231 cells (FIG. 7D). These findings show that MAPK activation by BCI-215 is not a general stress response.

Inhibition of p38, but not ERK or JNK/SAPK, Partially Reverses BCI-215 Toxicity.

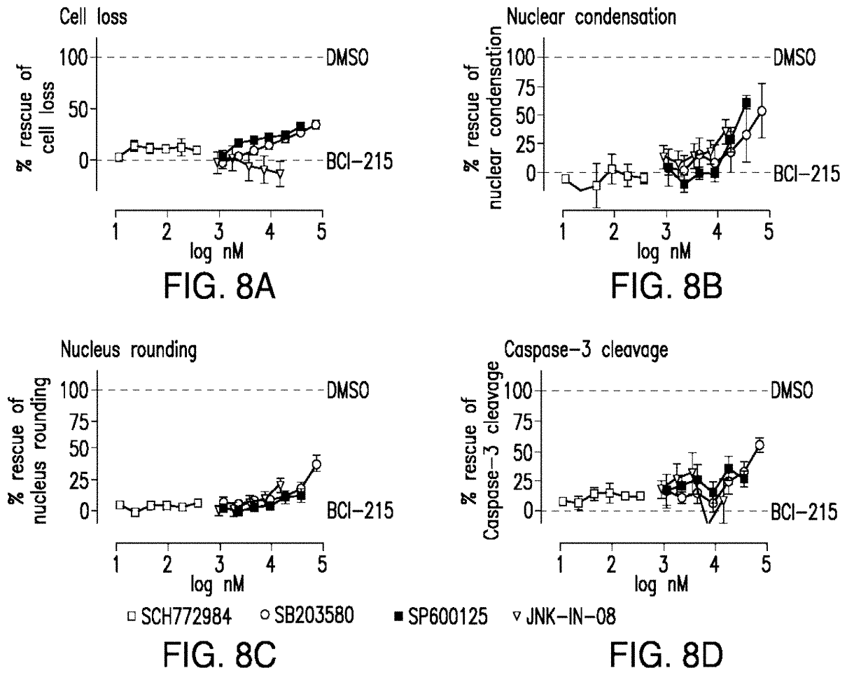

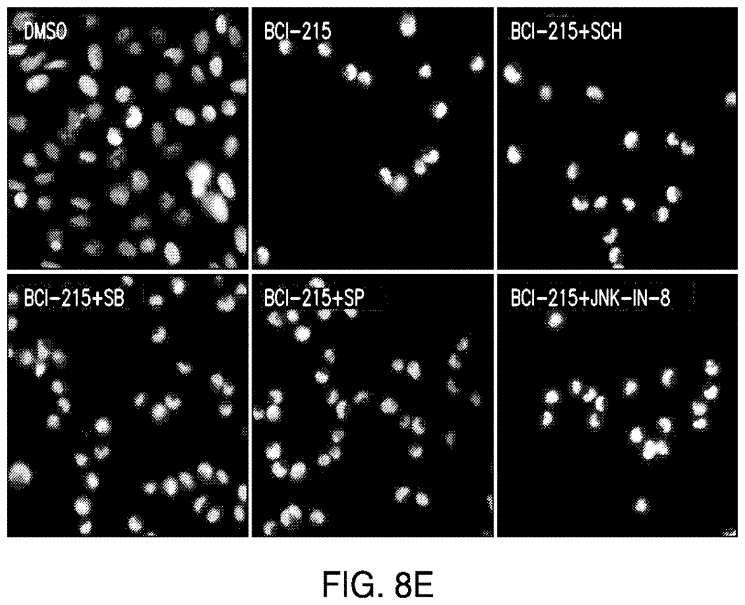

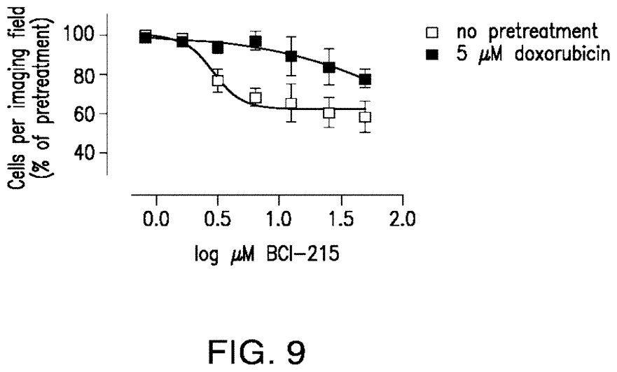

To examine whether activation of MAPK signaling contributed to BCI-215 cytotoxicity, MAPK inhibitors were used to probe pathway involvement, since all three MAPKs can autophosphorylate (33-35). Cells were treated on two halves of a 384 well plate with identical concentration gradients of selective ERK, JNK, and p38 inhibitors (SCH772984, JNK-IN-8, and SB203580), and a multitargeted inhibitor of INK (SP600125), respectively, bracketed around published concentrations reported to inhibit cellular MAPK activity (SCH771984, 30 nM (33); SP600125, 10 μM (34); SB203580, 10 μM (35), and JNK-IN-8, 0.5 μM (36). After a 30-min pre-incubation (3 hours for INK-IN-8), one half of the microplate was treated with vehicle (DMSO), the other with a pro-apoptotic concentration of BCI-215 (25 μM). After a 24-hour exposure, plates were stained with Hoechst 33342, and analyzed for cell numbers and nuclear morphology on the ArrayScan II. Plates were subsequently immunostained with a cleaved caspase-3 antibody. FIG. 5 shows that p38 and nonselective INK inhibition partially reversed BCI-215-induced cell loss, nuclear morphology changes, and apoptosis (FIG. 8), whereas specific inhibition of ERK or INK had no effect. The partial rescue of toxicity indicates that either both p38 and INK inhibition are necessary for full reversal of toxicity, or that MAPK-unrelated pathways also contribute to BCI-215 cytotoxicity. Next, the BCI-215 toxicity under conditions reported to downregulate DUSP1/MKP-1 was assessed. MDA-MB-231 cells carrying the GFP-labeled cytochrome C biosensor were pre-treated for 2-3 hours with doxorubicin, which downregulates DUSP1/MKP-1 by a transcriptional mechanism (29) but does not cause morphological changes and apoptosis similar to BCI-215 until after several days of exposure. Cells were subsequently treated with BCI-215 and analyzed for cell numbers 24 hours thereafter. FIG. 9 shows that doxorubicin reduced the toxicity of BCI-215, consistent with prior observations that MKP inhibition synergizes with chemotherapeutic agents under conditions that elevate DUSP1/MKP-1 (7, 14).

### 6.3 Discussion

It has long been proposed that overexpression of DUSP-MKPs represents a dependency of cancer cells, but to date, efforts to target DUSP-MKPs with small molecules have failed. The druggability of DUSP-MKPs has been questioned based on the feature-poor nature of their catalytic site, sensitivity to oxidation, and a high degree of conservation between members of the DUSP-MKP family. It is also being argued that even if it were possible to selectively inhibit individual DUSP-MKPs, off-target effects would invariably pose a problem because of overlapping substrate specificities. Recent studies and findings presented here show that these views are too simplistic. BCI-215 inhibits at least two DUSPs and yet is completely devoid of normal cell and developmental toxicity. Because BCI-215's biological activities were not obscured by toxicity, this compound is the first to permit testing the hypothesis that it is possible to pharmacologically target DUSP-MKPs as a dependency of cancer cells. BCI-215 selectively killed cancer cells but spared cultured hepatocytes. In contrast to previously identified DUSP-MKP inhibitors, BCI-215 did not generate ROS. BCI-215 caused apoptosis but not primary necrosis, showing a physiologic form of cell kill that in clinical settings can avoid the complication of tumor lysis syndrome and resultant inactivation of immune cells (37).

BCI-215 sensitized cancer cells to LAK activity. The mechanisms for the remarkable shift in LAK potency are currently under investigation but are likely due to enhanced expression or secretion of stress ligands by treated cells, activating immune cells and causing immunogenic cell death (ICD) (26). The presence of immune cells in the tumor bed is one of the most powerful prognostic indicators of patient survival (38). Only a few chemotherapies induce ICD with different clinical outcomes (26). ICD involves induced expression of stress ligands on tumor cells (39), enabling recognition of tumor cells, facilitating enhanced interactions between tumor cells and immune effectors, release of IFN gamma and HMGB1, enhanced survival/autophagy in responding cells, and lytic elimination of tumor cells unable of responding temporally in an effective manner. Specific candidate mechanisms for ICD worthy of investigation are NKG2D (NK expressed molecule G2D, one of twelve “unique” NK receptors not expressed in lymphoblastoid cell lines) or STING (for stimulator of interferon genes). Innate immune cells (40) but also T-cells (41) express NKG2D as a stress receptor sensitive to stressed cells. NKG2D ligand expression is positively correlated with longer relapse-free period in breast cancer patients (42). Furthermore, the mechanism of chemotherapy induced stress ligand expression likely involves the STING pathway (43) induced by DNA damage or other means to activate STING. An alternative notion is that such chemotherapy promotes recognition through enhanced recognition of “altered self” with diminished expression of molecules in stressed cells (44).

BCI-215 sensitized cancer cells to LAK activity despite showing little cell lysis in two-dimensional culture. This shows that display of phosphatidylserine (Annexin V stain) and a relatively modest amount of secondary necrosis, which is necessary for soluble ligand release, are sufficient for the observed level of sensitization. Alternatively, cells grown in microenvironments that more closely resemble in vivo conditions can be more susceptible to BCI-215. Experiments in long-term (one week) three-dimensional matrigel culture documented that BCI-215 prevented colony outgrowth and resulted in much higher levels of cell lysis compared to short term monolayer culture. This opens up the possibility that BCI-215 could cause enhanced immunogenic cell death (ICD) in microenvironments more closely resembling the metastatic niche.

It is also possible to directly exploit DUSP-MKP inhibition to boost immune responses. In aging patients, inhibition of DUSP6/MKP-3 by BCI enhanced the activity of T-cells by restoring defective ERK signaling caused by increased DUSP6/MKP-3 expression (45). Thus, it is conceivable that BCI-215 could directly activate PBMCs or augment IL-2 activity (which is dependent on ERK activation).

The effects of BCI and BCI-215 are not limited to MDA-MB-231 cells. Both agents activate stress signaling in BT20 and HeLa cells. BCI has been tested in the NCI 60 cell line panel (NSC150117) with a mean GI50 of 1.84 μM and a preference for leukemia cells (last tested June 2016). Consistent with this, Müschen's group demonstrated BCI selectively induced cell death in patient-derived pre-B acute lymphoblastic leukemia (pre-B-ALL) cells, likely through inhibition of DUSP6/MKP3, which they showed to be essential for oncogenic transformation in mouse models of pre B-ALL (46).

To what extent the effects of BCI-215 on cancer cell toxicity are mediated by DUSPs can presently not be answered definitively but the present results are consistent with DUSP inhibition. Prior studies by the inventors show that BCI analogs are bona fide inhibitors of at least some DUSPs. BCI and BCI-215 override the effects of ectopic DUSP6/MKP-3 and DUSP1/MKP-1 expression in HeLa cells (12). In zebrafish embryos, BCI restores FGF target gene expression in the presence of overexpressed Dusp6 but not Dusp5 or sprouty (11). Thus, BCI-215 is a valuable, non-toxic chemical probe for specific DUSP-mediated biologies. In cancer cells, which express multiple, redundant DUSPs, evidence is indirect but most consistent with negative feedback inhibition. BCI-215 rapidly and persistently activated MAPKs, different from the fast but transient response of growth factors or the delayed but persistent response by radiation, death ligands (47), or doxorubicin (29), arguing against ligand-like or transcriptional mechanisms. BCI-215 also does not appear to be a general stress stimulus, as those are usually associated with ROS generation (32). Collectively, the results favor a catalytic mechanism involving elimination of negative feedback downstream of growth factor or stress receptors.

BCI-215 causes a toxicity phenotype similar that of DUSP1/MKP-1 knockdown (e.g., apoptosis and reduced motility (10)). This renders target involvement studies based on simple genetic deletions non-definitive, as either sensitization or desensitization could be interpreted as consistent with DUSP inhibition. BCI-215 toxicity was also assessed in the presence of doxorubicin, which downregulates DUSP1/MKP-1 within hours by a transcriptional mechanism but does not cause morphological changes and apoptotic death until much later (29), and found that BCI-215 toxicity was reduced (FIG. 9).

BCI-215 activated kinases upstream of MAPKs. This result shows that in cancer cells, BCI-215 can have polypharmacological activities. Drug polypharmacology offers opportunities for discovery. An analysis of known drug-target interactions for agents in DrugBank reveals an average of 3.35 target interactions per drug, and 4.50 drug interactions per target (48). The predicted number of interactions is at least an order of magnitude higher (49), suggesting promiscuity is inevitable. It could be argued, as it has been for other agents in heterogeneous, complex diseases (50) that the biological activities of BCI-215 are a result of polypharmacology that likely cannot be recapitulated by single target inhibition. The combination of increased MAPK signaling, lack of toxicity, and a profile of immune cell sensitization distinct from known antineoplastics, may encourage investigation of polypharmacology, not only to advance BCI-215 as a complement to cancer immunotherapy, but also to maybe uncover novel mechanisms for immunogenic cell kill. This may require a comprehensive analysis of BCI-215's molecular mechanism(s) of action through an array of orthogonal assays including phosphoproteome profiling, target engagement studies, chemical proteomics, and functional genomics.

### 7. REFERENCES

1. Kidger A M, Keyse S M. The regulation of oncogenic Ras/ERK signalling by dual-specificity mitogen activated protein kinase phosphatases (MKPs). Semin Cell Dev Biol. 2016; 50:125-32.
2. Farooq A, Zhou M M. Structure and regulation of MAPK phosphatases. Cell Signal. 2004; 16:769-79.
3. Keyse S M. Dual-specificity MAP kinase phosphatases (MKPs) and cancer. Cancer Metast Rev. 2008; 27:253-61.
4. Nunes-Xavier C, Roma-Mateo C, Rios P, Tarrega C, Cejudo-Marin R, Tabernero L, et al. Dual-specificity MAP kinase phosphatases as targets of cancer treatment. Anticancer Agents Med Chem. 2011; 11:109-32.
5. Denkert C, Schmitt W D, Berger S, Reles A, Pest S, Siegert A, et al. Expression of mitogen-activated protein kinase phosphatase-1 (MKP-1) in primary human ovarian carcinoma. International Journal of Cancer. 2002; 102:507-13.
6. Montagut C, Iglesias M, Arumi M, Bellosillo B, Gallen M, Martinez-Fernandez A, et al. Mitogen-activated protein kinase phosphatase-1 (MKP-1) impairs the response to anti-epidermal growth factor receptor (EGFR) antibody cetuximab in metastatic colorectal cancer patients. Br J Cancer. 2010; 102:1137-44.
7. Wu W, Pew T, Zou M, Pang D, Conzen S D. Glucocorticoid receptor-induced MAPK phosphatase-1 (MPK-1) expression inhibits paclitaxel-associated MAPK activation and contributes to breast cancer cell survival. J Biol Chem. 2005; 280:4117-24.
8. Liu F, Gore A J, Wilson J L, Korc M. DUSP1 is a novel target for enhancing pancreatic cancer cell sensitivity to gemcitabine. PLoS One. 2014; 9:e84982.
9. Small G W, Shi Y Y, Higgins L S, Orlowski R Z. Mitogen-activated protein kinase phosphatase-1 is a mediator of breast cancer chemoresistance. Cancer Res. 2007; 67:4459-66.
10. Moncho-Amor V, Ibanez de Caceres I, Bandres E, Martinez-Poveda B, Orgaz J L, Sanchez-Perez I, et al. DUSP1/MKP1 promotes angiogenesis, invasion and metastasis in non-small-cell lung cancer. Oncogene. 2011; 30:668-78.
11. Molina G, Vogt A, Bakan A, Dai W, de Oliveira P Q, Znosko W, et al. Zebrafish chemical screening reveals an inhibitor of Dusp6 that expands cardiac cell lineages. Nat Chem Biol. 2009; 6:680-7.
12. Korotchenko V N, Saydmohammed M, Vollmer L L, Bakan A, Sheetz K, Debiec K T, et al. In vivo structure-activity relationship studies support allosteric targeting of a dual specificity phosphatase. Chembiochem. 2014; 15:1436-45.
13. Vogt A, Tamewitz A, Skoko J, Sikorski R P, Giuliano K A, Lazo J S. The benzo (c) phenanthridine alkaloid, sanguinarine, is a selective, cell-active inhibitor of mitogen-activated protein kinase phosphatase-1. J Biol Chem. 2005; 280:19078-86.
14. Vogt A, McDonald P R, Tamewitz A, Sikorski R P, Wipf P, Skoko J J, 3rd, et al. A cell-active inhibitor of mitogen-activated protein kinase phosphatases restores paclitaxel-induced apoptosis in dexamethasone-protected cancer cells. Mol Cancer Ther. 2008; 7:330-40.
15. McQueen C A. Isolation and culture of hepatocytes from different laboratory species. In: Tyson C A, Frazier J M, editors. Methods in toxicology. San Diego: Academic Press; 1993. p. 255-61.
16. Buchser W J, Laskow T C, Pavlik P J, Lin H M, Lotze M T. Cell-mediated autophagy promotes cancer cell survival. Cancer Res. 2012; 72:2970-9.
17. Senutovitch N, Vernetti L, Boltz R, DeBiasio R, Gough A, Taylor D L. Fluorescent protein biosensors applied to microphysiological systems. Exp Biol Med (Maywood). 2015; 240:795-808.
18. Pereira C V, Nadanaciva S, Oliveira P J, Will Y. The contribution of oxidative stress to drug-induced organ toxicity and its detection in vitro and in vivo. Expert Opin Drug Metab Toxicol. 2012; 8:219-37.
19. Seth D, Rudolph J. Redox regulation of MAP kinase phosphatase 3. Biochemistry. 2006; 45:8476-87.
20. Bova M P, Mattson M N, Vasile S, Tam D, Holsinger L, Bremer M, et al. The oxidative mechanism of action of ortho-quinone inhibitors of protein-tyrosine phosphatase alpha is mediated by hydrogen peroxide. Arch Biochem Biophys. 2004; 429:30-41.
21. Debiton E, Madelmont J C, Legault J, Barthomeuf C. Sanguinarine-induced apoptosis is associated with an early and severe cellular glutathione depletion. Cancer Chemotherapy & Pharmacology. 2003; 51:474-82.
22. Shibue T, Brooks M W, Inan M F, Reinhardt F, Weinberg R A. The outgrowth of micrometastases is enabled by the formation of filopodium-like protrusions. Cancer Discov. 2012; 2:706-21.
23. Elmore S. Apoptosis: a review of programmed cell death. Toxicol Pathol. 2007; 35:495-516.
24. Topalian S L, Drake C G, Pardoll D M. Immune checkpoint blockade: a common denominator approach to cancer therapy. Cancer Cell. 2015; 27:450-61.
25. Lotfi R, Kaltenmeier C, Lotze M T, Bergmann C. Until Death Do Us Part: Necrosis and Oxidation Promote the Tumor Microenvironment. Transfus Med Hemother. 2016; 43:120-32.
26. Kroemer G, Galluzzi L, Kepp O, Zitvogel L. Immunogenic cell death in cancer therapy. Annu Rev Immunol. 2013; 31:51-72.
27. Wennerberg E, Sarhan D, Carlsten M, Kaminskyy V O, D'Arcy P, Zhivotovsky B, et al. Doxorubicin sensitizes human tumor cells to NK cell- and T-cell-mediated killing by augmented TRAIL receptor signaling. Int J Cancer. 2013; 133:1643-52.
28. Yamaue H, Tanimura H, Noguchi K, Iwahashi M, Tsunoda T, Tani M, et al. Cisplatin treatment renders tumor cells more susceptible to attack by lymphokine-activated killer cells. J Clin Lab Immunol. 1991; 35:165-70.
29. Small G W, Somasundaram S, Moore D T, Shi Y Y, Orlowski R Z. Repression of mitogen-activated protein kinase (MAPK) phosphatase-1 by anthracyclines contributes to their antiapoptotic activation of p44/42-MAPK. J Pharmacol Exp Ther. 2003; 307:861-9.
30. Zheng C F, Guan K L. Cloning and characterization of two distinct human extracellular signal-regulated kinase activator kinases, MEK1 and MEK2. J Biol Chem. 1993; 268:11435-9.
31. Brancho D, Tanaka N, Jaeschke A, Ventura J J, Kelkar N, Tanaka Y, et al. Mechanism of p38 MAP kinase activation in vivo. Genes Dev. 2003; 17:1969-78.
32. McCubrey J A, Lahair M M, Franklin R A. Reactive oxygen species-induced activation of the MAP kinase signaling pathways. Antioxid Redox Signal. 2006; 8:1775-89.
33. Morris E J, Jha S, Restaino C R, Dayananth P, Zhu H, Cooper A, et al. Discovery of a novel ERK inhibitor with activity in models of acquired resistance to BRAF and MEK inhibitors. Cancer Discov. 2013; 3:742-50.
34. Mingo-Sion A M, Marietta P M, Koller E, Wolf D M, Van Den Berg C L. Inhibition of JNK reduces G2/M transit independent of p53, leading to endoreduplication, decreased proliferation, and apoptosis in breast cancer cells. Oncogene. 2004; 23:596-604.
35. Kim L, Del Rio L, Butcher B A, Mogensen T H, Paludan S R, Flavell R A, et al. p38 MAPK autophosphorylation drives macrophage IL-12 production during intracellular infection. J Immunol. 2005; 174:4178-84.
36. Zhang T, Inesta-Vaquera F, Niepel M, Zhang J, Ficarro S B, Machleidt T, et al. Discovery of potent and selective covalent inhibitors of JNK. Chem Biol. 2012; 19:140-54.
37. Howard S C, Jones D P, Pui C-H. The Tumor Lysis Syndrome. The New England Journal of Medicine. 2011; 364:1844-54.
38. Galon J, Costes A, Sanchez-Cabo F, Kirilovsky A, Mlecnik B, Lagorce-Pages C, et al. Type, density, and location of immune cells within human colorectal tumors predict clinical outcome. Science. 2006; 313:1960-4.
39. Miyashita T, Miki K, Kamigaki T, Makino I, Nakagawara H, Tajima H, et al. Low-dose gemcitabine induces major histocompatibility complex class I-related chain AB expression and enhances an antitumor innate immune response in pancreatic cancer. Clin Exp Med. 2015.
40. Feng H, Dong Y, Wu J, Qiao Y, Zhu G, Jin H, et al. Epirubicin pretreatment enhances NK cell-mediated cytotoxicity against breast cancer cells in vitro. Am J Transl Res. 2016; 8:473-84.
41. Deng L, Liang H, Xu M, Yang X, Burnette B, Anna A, et al. STING-Dependent Cytosolic DNA Sensing Promotes Radiation-Induced Type I Interferon-Dependent Antitumor Immunity in Immunogenic Tumors. Immunity. 2014; 41:843-52.
42. de Kruijf E M, Sajet A, van Nes J G, Putter H, Smit V T, Eagle R A, et al. NKG2D ligand tumor expression and association with clinical outcome in early breast cancer patients: an observational study. BMC Cancer. 2012; 12:24.
43. Woo S R, Fuertes M B, Corrales L, Spranger S, Furdyna M J, Leung M Y, et al. STING-dependent cytosolic DNA sensing mediates innate immune recognition of immunogenic tumors. Immunity. 2014; 41:830-42.
44. Fine J H, Chen P, Mesci A, Allan D S, Gasser S, Raulet D H, et al. Chemotherapy-induced genotoxic stress promotes sensitivity to natural killer cell cytotoxicity by enabling missing-self recognition. Cancer Res. 2010; 70:7102-13.
45. Li G, Yu M, Lee W W, Tsang M, Krishnan E, Weyand C M, et al. Decline in miR-181a expression with age impairs T cell receptor sensitivity by increasing DUSP6 activity. Nat Med. 2012; 18:1518-24.
46. Shojaee S, Caeser R, Buchner M, Park E, Swaminathan S, Hurtz C, et al. Erk Negative Feedback Control Enables Pre-B Cell Transformation and Represents a Therapeutic Target in Acute Lymphoblastic Leukemia. Cancer Cell. 2015; 28:114-28.
47. Dhanasekaran D N, Reddy E P. JNK signaling in apoptosis. Oncogene. 2008; 27:6245-51.
48. Cobanoglu M C, Liu C, Hu F, Oltvai Z N, Bahar I. Predicting drug-target interactions using probabilistic matrix factorization. J Chem Inf Model. 2013; 53:3399-409.
49. Zhou H, Gao M, Skolnick J. Comprehensive prediction of drug-protein interactions and side effects for the human proteome. Sci Rep. 2015; 5:11090.
50. Roth B L, Sheffler D J, Kroeze W K. Magic shotguns versus magic bullets: selectively non-selective drugs for mood disorders and schizophrenia. NatRevDrug Discov. 2004; 3:353-9.
51. Joy M E, Vollmer L L, Hulkower K, Stern A M, Peterson C K, Boltz R C, et al. A high-content, multiplexed screen in human breast cancer cells identifies profilin-1 inducers with anti-migratory activities. PLoS One. 2014; 9:e88350.
52. Senutovitch N, Vernetti L, Boltz R, DeBiasio R, Gough A, Taylor D L. Fluorescent protein biosensors applied to microphysiological systems. Exp Biol Med (Maywood). 2015; 240:795-808.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the systems and methods of the disclosed subject matter without departing from the spirit or scope of the disclosed subject matter. Thus, it is intended that the disclosed subject matter include modifications and variations that are within the scope of the appended claims and their equivalents.

Various patents and patent applications are cited herein, the contents of which are hereby incorporated by reference herein in their entireties.

1. A method of treating cancer in a subject comprising administering, to the subject, (i) an amount of a dual specificity mitogen-activated protein kinase phosphatase (DUSP-MKP) inhibitor that sensitizes cancer cells to immune cell killing, and (ii) an agent that promotes a cell-mediated anti-cancer immune response in the subject.

2. The method of claim 1, wherein the cancer comprises cells that are resistant to an inhibitor monotherapy.

3. The method of claim 1, wherein the dual specificity mitogen-activated protein kinase phosphatase (DUSP-MKP) inhibitor inhibits DUSP6-induced dephosphorylation of extracellular signal-related kinase (ERK).

4. The method of claim 1, wherein the dual specificity mitogen-activated protein kinase phosphatase (DUSP-MKP) inhibitor is BCI-215 or an analog or prodrug thereof.

5. The method of claim 1, wherein the agent that promotes a cell-mediated anti-cancer immune response is an antibody directed toward an antigen selected from the group consisting of CTLA-4, PD-1, PD-L1, CD52, and CD20.

6. The method of claim 1, wherein the agent that promotes a cell-mediated anti-cancer immune response comprises immune cells selected from the group consisting of natural killer cells and dendritic cells, wherein the immune cells are activated in vitro and introduced to the subject.

7. (canceled)

8. The method of claim 1, wherein the agent that promotes a cell-mediated anti-cancer immune response comprises T cells, wherein the T cells are genetically modified to target cancer cells and introduced to the subject.

9. (canceled)

10. The method of claim 1, wherein the agent that promotes a cell-mediated anti-cancer immune response comprises interleukin-2 (IL-2)-activated peripheral blood mononuclear cells (PBMCs).

11. (canceled)

12. The method of claim 1, wherein the agent that promotes a cell-mediated anti-cancer immune response comprises a cytokine selected from interleukin-2 and interferon-α.

13. The method of claim 1, wherein the dual specificity mitogen-activated protein kinase phosphatase (DUSP-MKP) inhibitor and the agent that promotes a cell-mediated anti-cancer immune response are administered concurrently or sequentially.

14. (canceled)

15. The method of claim 1, wherein the dual specificity mitogen-activated protein kinase phosphatase (DUSP-MKP) inhibitor is 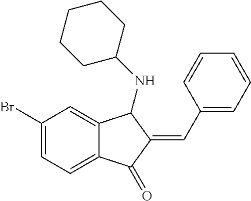 [SMILES: O=C1/C(=C/c2ccccc2)C(NC2CCCCC2)c2cc(Br)ccc21]
or an analog or prodrug thereof.

16. A dual specificity mitogen-activated protein kinase phosphatase (DUSP-MKP) inhibitor, for use in a method of treating cancer in a subject comprising administering, to the subject, (i) an amount of a dual specificity mitogen-activated protein kinase phosphatase (DUSP-MKP) inhibitor that sensitizes cancer cells to immune cell killing, and (ii) an agent that promotes a cell-mediated anti-cancer immune response in the subject.

17. The dual specificity mitogen-activated protein kinase phosphatase (DUSP-MKP) inhibitor of claim 16, wherein the cancer comprises cells that are resistant to an inhibitor monotherapy.

18. The dual specificity mitogen-activated protein kinase phosphatase (DUSP-MKP) inhibitor of claim 16, wherein the dual specificity mitogen-activated protein kinase phosphatase (DUSP-MKP) inhibitor inhibits DUSP6-induced dephosphorylation of extracellular signal-related kinase (ERK).

19. The dual specificity mitogen-activated protein kinase phosphatase (DUSP-MKP) inhibitor of claim 16, which is BCI-215 or an analog or prodrug thereof.

20. The dual specificity mitogen-activated protein kinase phosphatase (DUSP-MKP) inhibitor of claim 16 which is 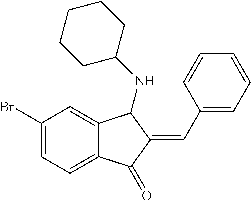 [SMILES: O=C1/C(=C/c2ccccc2)C(NC2CCCCC2)c2cc(Br)ccc21]
or an analog or prodrug thereof.

21. The dual specificity mitogen-activated protein kinase phosphatase (DUSP-MKP) inhibitor of claim 16, wherein the agent that promotes a cell-mediated anti-cancer immune response is an antibody directed toward an antigen selected from the group consisting of CTLA-4, PD-1, PD-L1, CD52, and CD20.

22. The dual specificity mitogen-activated protein kinase phosphatase (DUSP-MKP) inhibitor of claim 16, wherein the agent that promotes a cell-mediated anti-cancer immune response comprises immune cells selected from the group consisting of natural killer cells and dendritic cells, wherein the immune cells are activated in vitro and introduced to the subject.

23. (canceled)

24. The dual specificity mitogen-activated protein kinase phosphatase (DUSP-MKP) inhibitor of claim 16, wherein the agent that promotes a cell-mediated anti-cancer immune response comprises T cells, wherein the T cells are genetically modified to target cancer cells and introduced to the subject.

25. (canceled)

26. The dual specificity mitogen-activated protein kinase phosphatase (DUSP-MKP) inhibitor of claim 16, wherein the agent that promotes a cell-mediated anti-cancer immune response comprises interleukin-2 (IL-2)-activated peripheral blood mononuclear cells (PBMCs).

27. (canceled)

28. The dual specificity mitogen-activated protein kinase phosphatase (DUSP-MKP) inhibitor of claim 16, wherein the agent that promotes a cell-mediated anti-cancer immune response comprises a cytokine selected from interleukin-2 and interferon-α.

29. The dual specificity mitogen-activated protein kinase phosphatase (DUSP-MKP) inhibitor of claim 16, wherein the dual specificity mitogen-activated protein kinase phosphatase (DUSP-MKP) inhibitor and the the agent that promotes a cell-mediated anti-cancer immune response are administered either concurrently or sequentially.

30-31. (canceled)

32. A method for reducing cancer cell proliferation or promoting cancer cell death or inhibiting cancer cell metastasis in a subject in need thereof comprising administering to the subject an effective amount of (i) a first agent comprising a DUSP-MKP inhibitor that sensitizes cancer cells to immune cell killing and (ii) a second agent that promotes immune cell killing.

33. (canceled)

34. The method of claim 32, wherein the DUSP-MKP inhibitor is: 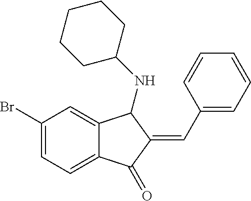 [SMILES: O=C1/C(=C/c2ccccc2)C(NC2CCCCC2)c2cc(Br)ccc21]
or an analog thereof, in an amount effective to increase levels of phosphorylated ERK or to decrease levels of de-phosphorylated ERK in the cancer cell and sensitize the cancer cell to immune cell killing.

35-36. (canceled)

37. A kit comprising: (i) one or more agent that can (a) decrease/inhibit the activity of DUSP6-MKP; (b) decrease the activity DUSP6 and DUSP1; (c) sensitize cancer cells to immune cell killing; and (d) reduce or inhibit cancer cell and/or tumor cell growth and (ii) one or more agent that can promote immune cell killing.

38. (canceled)
